(19) United States
(12) Patent Application Publication
(10) Pub. No.: US 2025/0143990 A1
(43) Pub. Date: May 8, 2025

(54) SKIN MICROBIOME FRIENDLY SUNSCREENS

(71) Applicant: BASF SE, Ludwigshafen am Rhein (DE)

(72) Inventors: Myriam SOHN, Grenzach-Wyhlen (DE); Marcel SCHNYDER, Grenzach-Wyhlen (DE); Juergen WIETHAN, Grenzach-Wyhlen (DE); Annette MEHLING, Düsseldorf-Holthausen (DE)

(21) Appl. No.: 18/727,765

(22) PCT Filed: Sep. 21, 2022
(86) PCT No.: PCT/EP2022/076204

§ 371 Date: Jul. 10, 2024

(30) Foreign Application Priority Data
Jan. 11, 2022 (EP) 22150982.1

(51) Int. Cl. A61K 8/41; A61Q 17/04

(52) U.S. Cl. CPC A61K 8/415; A61Q 17/04; A61K 2800/30; A61K 2800/48; A61K 2800/5922

(57) ABSTRACT

The present invention relates to microbiome friendly sunscreen or daily care compositions comprising certain UV filters such as hexyl 2-[4-(diethylamino)-2-hydroxybenzoyl]benzoate.

The present invention relates to the use of a sunscreen or daily care composition comprising certain UV filters for increasing the compatibility of the sunscreen or daily composition with skin microbiome, preferably human skin microbiome. Further, the invention is directed to specific sunscreen or daily care, which are suitable for the microbiome friendly application. Finally, the invention relates to the use of a composition (A) comprising certain UV filters for producing a sunscreen or daily care composition, which is compatible with the skin microbiome, preferably human skin microbiome.

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter (also referred to as UVA filter), UV-B filter (also referred to as UVB filter) and broadband filters. These filters are added to sunscreen or daily care compositions. The UV filters are either organic or inorganic, particulate or non-particulate compounds, of which all have a high absorption efficacy in the UV-light range. In general, UV light can be divided into UV-A radiation (320-400 nm) and UV-B radiation (290-320 nm). Depending on the position of the absorption maxima, UV filters are divided into UV-A and UV-B filters. In case a UV filter absorbs both, UV-A and UV-B light, it is referred to as a broadband absorber.

Since 2006, the EU commission has recommended that all sunscreen or daily care compositions should have a UV-A protection factor, which is at least one third of the labelled sun protection factor (SPF), wherein the sun protection factor refers mainly to the UV-B protection.

The human skin comprises millions of bacteria, fungi, and viruses that compose the skin microbiome. These organisms have essential roles in e.g. the protection against invading pathogens. When the barrier, which is provided by these organisms, is broken, humans are vulnerable to invading pathogens. It is known that e.g. compounds comprised in cosmetic products tend to break said barrier.

Even if main marketed products are providing both UVB and UVA protection according to the current standards, there is a need for improvement of the skin microbiome friendliness of e.g. sunscreens. Skin microbiome friendliness is an important performance indicator for end consumers, since users exemplarily need UV protection while parallel remaining a well-balanced skin microbiome. In addition, a lot of sunscreen or daily care compositions contain UV filters and other ingredients such as dispersing agents, which are frequently under discussion due to some concerns on human safety and skin sensitization potential, especially for a daily use, although they are approved for being used in leave-on compositions.

Against this background, there is an ongoing need for sunscreen or daily care compositions for the daily use, which provide efficient sun protection and provide a well-balanced skin microbiome. In particular, it was an object of the present invention to provide a sunscreen or daily care composition comprising a microbiome friendly UV filter. It was further an object of the present invention to provide a microbiome friendly sunscreen or daily care composition. Further, it was an object to provide a sunscreen or daily care composition comprising a UV filter for use in a method of increasing the compatibility of the sunscreen or daily care composition comprising with the skin microbiome. Further, it was an object of the present invention to provide a method of producing a skin microbiome friendly sunscreen or daily care composition. Finally, it was an object of the present invention to provide a composition (A) comprising a UV filter for the method of producing a skin microbiome friendly sunscreen or daily care composition.

It has surprisingly been found that at least one of these objects can be achieved by the sunscreen or daily care compositions and/or the compositions according to the present invention.

Thus, according to one aspect, the present invention relates to the use of a sunscreen or daily care composition comprising a UV filter selected from the group consisting of hexyl 2-[4-(diethylamino)-2-hydroxybenzoyl]benzoate (INCI diethylamino hydroxybenzoyl hexyl benzoate, also known as DHHB), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine, also known as BEMT), Phenyl-3H-benzimidazole-5-sulfonic acid (INCI Phenylbenzimidazole Sulfonic Acid, also known as PBSA), Terephthalylidene Dicamphor Sulfonic Acid (also known as TDSA), drometrizole trisiloxane (also known as DTS) and mixtures thereof, for increasing the compatibility of the sunscreen or daily composition with skin microbiome, preferably human skin microbiome.

In the following, preferred embodiments of the above use are described in further detail. It is to be understood that each preferred embodiment is relevant on its own as well as in combination with other preferred embodiments.

In a preferred embodiment A1a of the first aspect, the sunscreen or daily care composition comprises hexyl 2-[4-(diethylamino)-2-hydroxybenzoyl]benzoate (INCI diethylamino hydroxybenzoyl hexyl benzoate) and optionally further comprising 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid, and/or drometrizole trisiloxane.

In a preferred embodiment A1 b of the first aspect, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and optionally hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), Terephthalylidene Dicamphor Sulfonic Acid, and/or drometrizole trisiloxane.

In a preferred embodiment A1c of the first aspect, the sunscreen or daily care composition comprises 2-Phenyl-3H-benzimidazole-5-sulfonic acid (INCI Phenylbenzimidazole Sulfonic Acid) and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), Terephthalylidene Dicamphor Sulfonic Acid, and/or drometrizole trisiloxane.

In a preferred embodiment A1d of the first aspect, the sunscreen or daily care composition comprises Terephthalylidene Dicamphor Sulfonic Acid and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), and/or drometrizole trisiloxane.

In a preferred embodiment A1e of the first aspect, the sunscreen or daily care composition comprises drometrizole trisiloxane and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), and/or Terephthalylidene Dicamphor Sulfonic Acid.

In a preferred embodiment A2 of the first aspect, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the population of bacteria selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium tuberculostearicum, Corynebacterium fastidiosum, Corynebacterium simulans, Corynebacterium amycolatum Corynebacterium xerosis, Streptococcus mitis, Streptococcus sanguinis, Streptococcus oralis, Brevibacterium spp., Pseudomonas spp., and mixtures thereof, preferably Staphylococcus epidermidis, on the skin and/or
increases the compatibility of the sunscreen or daily care composition with the population of fungi selected from the group consisting of Malassezia furfur; Malassezia restricta, Malassezia globosa, Candida spp., and mixtures thereof on the skin.

In a preferred embodiment A3 of the first aspect, the sunscreen or daily care composition further comprises at least one additional UVB filter, preferably selected from the group consisting of 4,4′,4″-(1,3,5-triazin-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), dimethicone diethyl benzylmalonate (INCI polysilicone-15), and mixtures thereof, and in particular selected from the group consisting of 4,4′,4″-(1,3,5-triazin-2,4,6-triyltriimino)tris-benzoic acid-tris(2-ethylhexyl)ester (INCI ethylhexyl triazone or EHT), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), and mixtures thereof.

In a preferred embodiment A4 of the first aspect, the sunscreen or daily care composition further comprises at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine) preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine).

In a preferred embodiment A5 of the first aspect, the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside) and/or wherein the at least one organic micronized UV filter is dispersed in polyglycerol monoalkyl ester.

In a preferred embodiment A6 of the first aspect, the sunscreen or daily care composition further comprises at least one additional UVA filter, preferably selected from the group consisting of disodium phenyl dibenzimidazole tetrasulfonate, 1-(4-(1,1-dimethylethyl)phenyl)-3-(4-methoxyphenyl)propane-1,3-dione (INCI Butyl Methoxydibenzoylmethane), and mixtures thereof, in particular 1-(4-(1,1-dimethylethyl)phenyl)-3-(4-methoxyphenyl)propane-1,3-dione (INCI Butyl Methoxydibenzoylmethane).

In a preferred embodiment A7 of the first aspect, the sunscreen or daily care composition does not contain zinc oxide (ZnO).

In a preferred embodiment A8 of the first aspect, the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10 wt.-%, preferably from 0.5 to 8 wt.-%, and in particular from 0.5 to 5 wt.-%, based on the total weight of the sunscreen or daily care composition.

In a preferred embodiment A9 of the first aspect, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7 wt.-%, preferably from 0.5 to 4 wt. %, based on the total weight of the sunscreen or daily care composition and/or
2-Phenyl-3H-benzimidazole-5-sulfonic acid (INCI Phenylbenzimidazole Sulfonic Acid) in an amount of from 0.1 to 6 wt.-%, preferably from 0.5 to 4 wt.-%, based on the total weight of the sunscreen or daily care composition and/or
Terephthalylidene Dicamphor Sulfonic Acid (TDSA) in an amount of from 0.1 to 10 wt.-%, preferably from 0.2 to 5 wt.-%, based on the total weight of the sunscreen or daily care composition, and/or
drometrizole trisiloxane (DTS) in an amount of from 0.1 to 10 wt.-%, preferably from 0.2 to 6 wt.-%, based on the total weight of the sunscreen or daily care composition.

In a preferred embodiment A10 of the first aspect, the sunscreen or daily care composition comprises at least one organic micronized UV filter in an amount of from 0.1 to 10 wt. %, preferably from 0.5 to 5 wt.-%, based on the total weight of the sunscreen or daily care composition.

In a preferred embodiment A11 of the first aspect, the sunscreen or daily care composition comprises 4,4′,4″-(1,3,5-triazin-2,4,6-triyltriimino)tris-benzoic acid-tris(2-ethylhexyl)ester (INCI ethylhexyl triazone) in an amount of from 0.1 to 5 wt.-%, preferably from 0.5 to 4 wt.-%, based on the total weight of the sunscreen or daily care composition.

In a preferred embodiment A12 of the first aspect, the sunscreen or daily care composition further comprises a dermatologically acceptable emulsifier, thickener, and/or emollient.

In a preferred embodiment A13 of the first aspect, the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and/or 3,3,5-Trimethylcyclohexyl 2-hydroxybenzoate (INCI homosalate).

In a second aspect, the present invention relates to a sunscreen or daily care composition comprising
0.1 to 10 wt.-%, preferably 0.5 to 5 wt.-%, of hexyl 2-[4-(diethylamino)-2-hydroxybenzoyl]benzoate (INCI diethylamino hydroxybenzoyl hexyl benzoate),
0.1 to 8 wt.-%, preferably 0.5 to 4 wt.-%, of 2-Phenyl-3H-benzimidazole-5-sulfonic acid (INCI Phenylbenzimidazole Sulfonic Acid),
0.1 to 5 wt.-%, preferably 0.5 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 5 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0 to 10 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 15 wt.-%, preferably 5 to 10 wt.-% of glycerol (also known as glycerine),
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), zinc oxide, and mixtures thereof.

In preferred embodiment B1 of the second aspect the 2,2′-methylene bis[6-(2Hbenzotriazol-2-yl)-4-(1,1,3,3-tetramethylbutyl)phenol](INCI Methylene bis-benzotriazolyl tetramethylbutylphenol)] is not dispersed in (3R,4S,5S,6R)-2-(Decyloxy)-6-(hydroxymethyl)tetrahydro-2H-Pyran-3,4,5-triol (INCI decyl glucoside).

In a third aspect, the present invention relates to the use of a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof for producing a sunscreen or daily care composition, which is compatible with the skin microbiome, preferably human skin microbiome.

In a preferred embodiment C1 of the third aspect, the sunscreen or daily care composition further comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or 2-Phenyl-3H-benzimidazole-5-sulfonic acid (INCI Phenylbenzimidazole Sulfonic Acid).

### DETAILED DESCRIPTION

Before describing in detail exemplary embodiments of the present invention, definitions which are important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±15%, preferably ±10%, more preferably ±5%, even more preferably ±2%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise”, “does not contain”, or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product, which absorbs and which may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen compositions, decorative preparations, and skin care preparations.

Suitable decorative preparations are, e.g., lipsticks, nail varnishes, eye shadows, mascaras, dry and moist make-up, rouge, powders, depilatory agents and suntan lotions.

Suitable skin care preparations are e.g., moisturizing, refining, and lifting preparations. The cited daily care compositions can be in the form of creams, ointments, pastes, foams, gels, lotions, powders, make-ups, sprays, sticks or aerosols.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “polarity index” refers to non-polar or polar oils. Non-polar oils are mainly based on hydrocarbons and lack an electronegative element, such as oxygen. In contrast, polar oils contain heteroatoms that differ in electronegativity, which results in a dipole moment. However, such oils are still insoluble in water, i.e. hydrophobic. The polarity index can be determined by measuring the interfacial tension between the respective oil and water.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at 370 nm.

Furthermore, in Europe, it is recommended by the European Commission that all sunscreen or cosmetic compositions should have a UV-A protection factor, which is at least one third of the labelled sun protection factor (SPF), e.g. if the sunscreen composition has an SPF of 30 the UVA protection factor has to be at least 10.

The term “administration” refers to the application of a sunscreen or daily care composition to the skin of a person.

Preferred embodiments regarding the sunscreen or daily care composition as well as the use thereof (e.g. in a method of increasing the compatibility of the sunscreen or daily composition with skin microbiome) and/or the use of a composition comprising DHHB for producing a sunscreen or daily care composition, which is compatible with the skin microbiome are described hereinafter. It is to be understood that the preferred embodiments of the invention are preferred alone or in combination with each other.

As indicated above, the present invention relates in one embodiment to the use of a sunscreen or daily care composition comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (also known as TDSA), drometrizole trisiloxane (also known as DTS), and mixtures thereof, for increasing the compatibility of the sunscreen or daily composition with skin microbiome, preferably human skin microbiome.

As above-mentioned the skin microbiome comprises numerous organisms, which are important for protecting e.g. mammal (such as humans) against pathogens. It is known that several compounds have a negative effect on said skin microbiome. It is to be understood that “increasing the compatibility of the sunscreen or daily composition with skin microbiome” according to the present invention denotes that the inventive compositions comprising one of the specific UV filters have a superior microorganism population over the exact same composition, which merely does not comprise the specific UV filters. Suitable representatives of a microorganism of the skin microbiome are e.g. Staphylococcus epidermidis, Micrococcus luteus, and Corynebacterium jeikeium.

In connection with the present invention, the following preferred embodiments regarding the sunscreen or daily care composition are relevant. It is to be understood that each preferred embodiment is relevant on its own as well as in combination with other preferred embodiments.

In a preferred embodiment, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the long-term resident human skin microbiota (also known as human skin microbiome) mainly consisting of gram-positive bacteria especially the general Staphylococcus, Corynebacterium, Propionibacterium, Micrococcus, Streptococcus, and Brevibacterium and gram-negative bacteria such as Pseudomonas, as well as yeasts such as the genera Malassezia (former Pityrosporum) or Candida. Especially of relevance for the healthy resident microflora are the species selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., Malassezia furfur, Malassezia restricta, Malassezia globosa, Candida spp., and mixtures thereof. It is especially preferred that the compatibility with Staphylococcus epidermidis, Micrococcus luteus, and/or Corynebacterium jeikeium, and in particular with Staphylococcus epidermidis, is increased.

In a preferred embodiment, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the population of bacteria selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., and mixtures thereof, on the skin. More preferably, the compatibility with the population of Staphylococcus epidermidis, Micrococcus luteus, and/or Corynebacterium jeikeium, and in particular of Staphylococcus epidermidis, is increased.

In this connection, the meaning of increasing the comparability with skin microbiome is illustrated with Staphylococcus epidermidis as follows:

It is to be understood that the compatibility with skin microbiome of the sunscreen or daily care composition (e.g. of the inventive composition comprising DHHB, BEMT, PBSA, TDSA and/or DTS) is increased if the population of Staphylococcus epidermidis determined after 24 hours is higher than the population of Staphylococcus epidermidis determined after 24 hours of the exact same composition, which merely does not comprise the specific UV filter (e.g. DHHB, BEMT, PBSA, TDSA and/or DTS). Further, it is to be understood that determining the respective Staphylococcus epidermidis population needs to be conducted in an equal manner.

Preferably, the determination of the population is conducted in vitro, wherein a subculture of Staphylococcus epidermidis on tryptic soy agar is suspended in tryptic soy broth and incubated at 36° C. for 24 h (overnight culture). The formulations to be tested are contaminated (TO) with the bacterial suspension (dilution of overnight culture of S. epidermidis in sodium chloride). At 24 h contact, each sample is spread out on the surface of tryptone soy agar plates and incubated for two days at 36° before microbial enumeration. Finally, the total number of colony forming units, cfu/mL, on each agar plate for all test conditions are counted.

In a preferred embodiment, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the population of fungi selected from the group consisting of Malassezia furfur; Malassezia restricta, Malassezia globosa, Candida spp., and mixtures thereof on the skin.

Preferably, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the population of species selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., Malassezia furfur, Malassezia restricta, Malassezia globosa, Candida spp., and mixtures thereof, more preferably selected from the group consisting of Staphylococcus epidermidis, Micrococcus luteus, and/or Corynebacterium jeikeium, and mixtures thereof, on the skin.

In a preferred embodiment, the sunscreen or daily care composition comprises hexyl 2-[4-(diethylamino)-2-hydroxybenzoyl]benzoate (INCI diethylamino hydroxybenzoyl hexyl benzoate).

In a preferred embodiment, the sunscreen or daily care composition comprises hexyl 2-[4-(diethylamino)-2-hydroxybenzoyl]benzoate (INCI diethylamino hydroxybenzoyl hexyl benzoate) and further 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or 2-Phenyl-3H-benzimidazole-5-sulfonic acid (INCI Phenylbenzimidazole Sulfonic Acid).

In a preferred embodiment, the sunscreen or daily care composition comprises hexyl 2-[4-(diethylamino)-2-hydroxybenzoyl]benzoate (INCI diethylamino hydroxybenzoyl hexyl benzoate) and optionally further 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), and/or drometrizole trisiloxane (DTS).

In a preferred embodiment, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and optionally hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), Terephthalylidene Dicamphor Sulfonic Acid, and/or drometrizole trisiloxane.

In a preferred embodiment, the sunscreen or daily care composition comprises 2-Phenyl-3H-benzimidazole-5-sulfonic acid (INCI Phenylbenzimidazole Sulfonic Acid) and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), Terephthalylidene Dicamphor Sulfonic Acid, and/or drometrizole trisiloxane.

In a preferred embodiment, the sunscreen or daily care composition comprises Terephthalylidene Dicamphor Sulfonic Acid and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), and/or drometrizole trisiloxane.

In a preferred embodiment, the sunscreen or daily care composition comprises drometrizole trisiloxane and optionally 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), hexyl 2-[4-(diethylamino)-2-hydroxybenzoyl]benzoate (INCI diethylamino hydroxybenzoyl hexyl benzoate), 2-Phenyl-3H-benzimidazole-5-sulfonic acid (INCI Phenylbenzimidazole Sulfonic Acid), and/or Terephthalylidene Dicamphor Sulfonic Acid.

It is particularly preferred that the sunscreen or daily care composition comprises hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and 2-Phenyl-3H-benzimidazole-5-sulfonic acid (INCI Phenylbenzimidazole Sulfonic Acid).

In a preferred embodiment, the sunscreen or daily care composition further comprises at least one additional UVB filter, preferably selected from the group consisting of 4,4′,4″-(1,3,5-triazin-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), dimethicone diethyl benzylmalonate (INCI polysilicone-15), and mixtures thereof, more preferably selected from the group consisting of 4,4′,4″-(1,3,5-triazin-2,4,6-triyltriimino)tris-benzoic acid-tris(2-ethylhexyl)ester (INCI ethylhexyl triazone or EHT), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), and mixtures thereof, and in particular 4,4′,4″-(1,3,5-triazin-2,4,6-triyltriimino)tris-benzoic acid-tris(2-ethylhexyl)ester (INCI ethylhexyl triazone or EHT).

In a preferred embodiment, the sunscreen or daily care composition further comprises at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine) preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine).

Preferably, the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside).

Preferably, the at least one organic micronized UV filter is dispersed in polyglycerol monoalkyl ester.

Preferably, the polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and mixtures thereof, more preferably wherein the at least one polyglycerol monoalkyl ester is decaglyceryl monolaurate (INCI Polyglyceryl-10 Laurate).

Polyglycerol monoalkyl esters having an HLB (hydrophilic-lipophilic balance) of 14.5 or more are preferable and having an HLB of 15 or more are more preferable. The HLB value can be determined by any means known in the art.

Polyglycerol monoalkyl esters having an HLB of less than 14.5 may take a longer time for dispersion of organic micronized UV filter (e.g. methylene bis-benzotriazolyl tetramethylbutylphenol) in water phase components. Examples of polyglycerol monoalkyl esters with a mean degree of polymerization of 5 or more and having an HLB of 14.5 or more may include decaglyceryl caprate, decaglyceryl monolaurate, decaglyceryl myristate, decaglyceryl oleate, decaglyceryl stearate, decaglyceryl isostearate, hexaglyceryl laurate, pentaglyceryl laurate, pentaglyceryl myristate, pentaglyceryl stearate, and pentaglyceryl oleate, and those having an HLB of 15 or more may include decaglyceryl caprate and decaglyceryl monolaurate.

Any known process suitable for the preparation of microparticles can be used for the preparation of the at least one organic micronized UV filter (e.g. methylene bis-benzotriazolyl tetramethylbutylphenol). Preferably, an organic compound insoluble in water (solubility lies in the ng/L range) methylene bis-benzotriazolyl tetramethylbutylphenol is present in the micronized state and is more preferably prepared by wet-milling processes.

As milling apparatus for the preparation of the at least one organic micronized UV filter (e.g. methylene bis-benzotriazolyl tetramethylbutylphenol) there may be used, for example, a jet mill, ball mill, vibratory mill or hammer mill, preferably a high-speed mixing mill.

The at least one organic micronized UV filter (e.g. Methylene bis-benzotriazolyl tetramethylbutylphenol) is preferably micronized in a ball mill to the desired mean particle size in the presence of water and of a polyglycerol monoalkyl ester such as Polyglyceryl-10 Laurate which acts as dispersing agent.

In a preferred embodiment, the sunscreen or daily care composition further comprises at least one additional UVA filter, preferably selected from the group consisting of disodium phenyl dibenzimidazole tetrasulfonate, 1-(4-(1,1-dimethylethyl)phenyl)-3-(4-methoxyphenyl)propane-1,3-dione (INCI Butyl Methoxydibenzoylmethane), and mixtures thereof, in particular 1-(4-(1,1-dimethylethyl)phenyl)-3-(4-methoxyphenyl)propane-1,3-dione (INCI Butyl Methoxydibenzoylmethane).

In a preferred embodiment, the sunscreen or daily care composition does not contain zinc oxide (ZnO). In a preferred embodiment, the sunscreen or daily care composition does not contain zinc derivative compounds. According to the present invention, zinc derivative compounds denote every compound that comprises a atom zinc.

In a preferred embodiment, the sunscreen or daily care composition does not contain a zinc salt. In a particular preferred embodiment, the sunscreen or daily care composition does not contain a zinc salt of pyrrolidone carboxylic acid.

In a preferred embodiment, the sunscreen or daily care composition does not contain parabens.

In a preferred embodiment, the sunscreen or daily care composition does not contain PVP (polyvinylpyrrolidone) polymers and VP (vinylpyrrolidone) derivatives such as vinylpyrrolidone/hexadecene copolymer, VP/Eicosene Copolymer, Triacontanyl PVP.

In a preferred embodiment, the sunscreen or daily care composition does not contain polymers based on acrylic acid such as for example not limited to compounds with the INCI carbomers, C10/30 alkyl acrylate crosspolymer.

In a preferred embodiment, the sunscreen or daily care composition further comprises C₂-C₁₂-alkandiol, preferably a 1,2-C₂-C₁₂-alkandiol or a 1,3-C₂-C₁₂-alkandiol, more preferably a 1,2-C₂-C₁₀-alkandiol or a 1,3-C₂-C₁₀-alkandiol, and in particular 1,2-C₂-C₁₀-alkandiol or 1,2-C₂-C₈-alkandiol. Preferably, the sunscreen or daily care composition comprises the C₂-C₁₂-alkandiol in an amount of from 0.5 to 10 wt.-%, more preferably from 1 to 8 wt.-%, based on the total weight of the sunscreen or daily care composition.

In a preferred embodiment, the sunscreen or daily care composition further comprises glycerol (also known as glycerine). Preferably, the sunscreen or daily care composition comprises the glycerol in an amount of from 3 to 15 wt.-%, more preferably from 4 to 10 wt.-%, even more preferably from 5 to 10 wt.-%, and in particular from 5 to 8 wt.-%, based on the total weight of the sunscreen or daily care composition.

In a preferred embodiment, the sunscreen or daily care composition comprises Sodium Stearoyl Glutamate in an amount of 0.1 to 2 wt.-%, preferably of 1 to 2 wt.-% or of 0.3 to 1 wt.-%, and in particular of 0.2 to 1 wt.-%, based on the total weight of the sunscreen or daily care composition.

In a preferred embodiment, the sunscreen or daily care composition comprises a monoester of stearic acid with glycerin (e.g. glyceryl stearate SE) in an amount of 1 to 4 wt.-%, preferably 1.5 to 3 wt.-%, based on the total weight of the sunscreen or daily care composition.

In a preferred embodiment, the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10 wt.-%, preferably from 0.5 to 8 wt.-%, and in particular from 0.5 to 5 wt.-%, based on the total weight of the sunscreen or daily care composition.

In a preferred embodiment, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 10 wt.-%, preferably from 0.1 to 7 wt.-%, and in particular from 0.5 to 4 wt. %, based on the total weight of the sunscreen or daily care composition.

In a preferred embodiment, the sunscreen or daily care composition comprises 2-Phenyl-3H-benzimidazole-5-sulfonic acid (INCI Phenylbenzimidazole Sulfonic Acid) in an amount of from 0.1 to 8 wt.-%, preferably from 0.1 to 6 wt.-%, and in particular from 0.5 to 4 wt.-%, based on the total weight of the sunscreen or daily care composition.

In a preferred embodiment, the sunscreen or daily care composition comprises Terephthalylidene Dicamphor Sulfonic Acid (TDSA) in an amount of from 0.1 to 10 wt.-%, preferably from 0.2 to 5 wt.-%, based on the total weight of the sunscreen or daily care composition.

In a preferred embodiment, the sunscreen or daily care composition comprises drometrizole trisiloxane (DTS) in an amount of from 0.1 to 10 wt.-%, preferably from 0.2 to 6 wt.-%, based on the total weight of the sunscreen or daily care composition.

In a preferred embodiment, the sunscreen or daily care composition comprises
hexyl 2-[4-(diethylamino)-2 hydroxybenzoyl]benzoate (INCI diethylamino hydroxybenzoyl hexyl benzoate) in an amount of from 0.1 to 8 wt.-%, preferably from 0.5 to 5 wt.-%;
2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7 wt.-%, preferably from 0.5 to 4 wt. %; and
2-Phenyl-3H-benzimidazole-5-sulfonic acid (INCI Phenylbenzimidazole Sulfonic Acid) in an amount of from 0.1 to 6 wt.-%, preferably from 0.5 to 4 wt.-%;
each based on the total weight of the sunscreen or daily care composition.

In a preferred embodiment, the sunscreen or daily care composition comprises at least one organic micronized UV filter in an amount of from 0.1 to 10 wt. %, preferably from 0.5 to 5 wt.-%, based on the total weight of the sunscreen or daily care composition.

In a preferred embodiment, the sunscreen or daily care composition comprises 4,4′,4″-(1,3,5-triazin-2,4,6-triyltriimino)tris-benzoic acid-tris(2-ethylhexyl)ester (INCI ethylhexyl triazone) in an amount of from 0.1 to 5 wt.-%, preferably from 0.5 to 4 wt.-%, based on the total weight of the sunscreen or daily care composition.

In a preferred embodiment, the sunscreen or daily care composition comprises 2,4,6-tris(biphenyl-4-yl)-1,3,5-triazine (INCI Tris-Biphenyl triazine) in an amount of from 0.1 to 5 wt.-%, preferably from 0.5 to 4 wt.-%, based on the total weight of the sunscreen or daily care composition.

In a preferred embodiment, the sunscreen or daily care composition further comprises 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), preferably in an amount of 0.1 to 10 wt.-%, more preferably in an amount of 0.2 to 6 wt.-%, and in particular in an amount of 0.5 to 4 wt.-%, based on the total weight of the sunscreen or daily care composition.

In a preferred embodiment, the sunscreen or daily care composition comprises DHHB and BEMT in a weight ratio of 5:1 to 1:4, preferably of 4:1 to 1:3, more preferably of 3:1 to 1:2, and in particular of 2:1 to 1:1.5.

In a preferred embodiment, the sunscreen or daily care composition comprises DHHB and PBSA in a weight ratio of 5:1 to 1:4, preferably of 4:1 to 1:3, more preferably of 3:1 to 1:2, and in particular of 2.5:1 to 1:1.5.

In a preferred embodiment, the sunscreen or daily care composition comprises DHHB and the sum of BEMT and PBSA in a weight ratio of 5:1 to 1:6, preferably of 4:1 to 1:5, more preferably of 2:1 to 1:4, and in particular of 1.5:1 to 1:2.5.

In a preferred embodiment, the sunscreen or daily care composition comprises DHHB and EHT in a weight ratio of 5:1 to 1:4, preferably of 4:1 to 1:3, more preferably of 3:1 to 1:2, and in particular of 2.5:1 to 1:1.5.

In a preferred embodiment, the sunscreen or daily care composition comprises DHHB and MBBT in a weight ratio of 5:1 to 1:4, preferably of 4:1 to 1:3, more preferably of 3:1 to 1:2, and in particular of 2.5:1 to 1:1.5.

In a preferred embodiment, the sunscreen or daily care composition comprises DHHB and glycerol in a weight ratio of 3:1 to 1:10, preferably of 2:1 to 1:9, more preferably of 1:1 to 1:9, and in particular of 1:2 to 1:7.

In a preferred embodiment, the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and/or homosalate.

Preferably, the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), and 3-Methylbutyl (2E)-3-(4-methoxyphenyl)prop-2-enoate (INCI Isoamyl p-Methoxycinnamate).

Preferably, the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and 3,3,5-Trimethylcyclohexyl 2-hydroxybenzoate (INCI homosalate).

In a preferred embodiment, the sunscreen or daily care composition further comprises a dermatologically acceptable emulsifier, thickener, and/or emollient.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C₁₂-C₁₅-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C₆-C₁₈ fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane.

Preferred thickeners include
fatty alcohols such as cetyl alcohol, cetearyl alcohol, behenyl alcohol, stearyl alcohol;
fatty acids such as stearic acid;
fatty acid esters such as myristyl stearate;
C18-38 Alkyl Hydroxystearoyl
waxes such as beeswax, carnauba wax, microcrystalline wax, ceresin, ozocerite, Oryza sativa Rice Bran Wax;
polysaccharides or derivatives such as xanthan gum, guar gum, agar gum, alginates, gellan gum, carraghenan, tara gum;
silicate derivatives such as magnesium silicates;
cellulose derivatives such as hydroxypropyl cellulose.

In one embodiment, the sunscreen or daily care composition further comprises a sensory enhancers, an adjuvants, a preservatives, and/or a perfumes.

Preferred sensory enhancers include
silica;
mica;
starch derivatives such as aluminum starch octenylsuccinate, Tapioca starch, Oryza sativa starch, Zea mays (corn) starch;
dimethicone derivatives;
boron nitride;
Perlite.

Preferred Humectant include
1,2 pentadiol;
1,2 hexandiol;
1,2 octandiol;
1,2 decandiol;
2-Methyl-1,3-propandiol.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the above preferred embodiments, it is to be understood that if the sunscreen or daily care composition comprises two or more additives (e.g. emulsifier, emollients, thickeners, sensory enhancers, adjuvants, preservatives, perfumes), combinations of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions or in the form of a sprayable product.

The above-outlined sunscreen and daily care compositions are in particular suitable for sensitive skin.

Further, the use of a sunscreen or daily care composition comprising 2,4,6-Tris(biphenyl-4-yl)-1,3,5-triazin (INCI Tris-biphenyl Triazine) for increasing the compatibility of the sunscreen or daily composition with skin microbiome is disclosed, wherein the 2,4,6-Tris(biphenyl-4-yl)-1,3,5-triazin (INCI Tris-biphenyl Triazine) is dispersed in polyglycerol monoalkyl ester. It is to be understood that the above-defined preferred embodiments also apply for this disclosure.

Further, a sunscreen or daily care composition comprising 2,4,6-Tris(biphenyl-4-yl)-1,3,5-triazin (INCI Tris-biphenyl Triazine) for use in a method of increasing the compatibility of the sunscreen or daily composition with skin microbiome is disclosed, wherein the 2,4,6-Tris(biphenyl-4-yl)-1,3,5-triazin (INCI Tris-biphenyl Triazine) is dispersed in polyglycerol monoalkyl ester. It is to be understood that the above-defined preferred embodiments also apply for this disclosure.

The present invention further relates to a sunscreen or daily care composition comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, for use in a method of increasing the compatibility of the sunscreen or daily composition with skin microbiome, preferably human skin microbiome. It is to be understood that above embodiments of the use aspect of the sunscreen or daily care composition are also applicable for this aspect, in particular regarding the microorganisms, the at least one additional UVB filter, at least one micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

The present invention further relates to a method of increasing the compatibility of a sunscreen or daily composition with skin microbiome, preferably human skin microbiome, the method comprising the step of mixing a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof with at least one other dermatologically acceptable ingredient.

It is to be understood that above embodiments of the use aspect of the sunscreen or daily care composition are also applicable for this aspect, in particular regarding the microorganisms, the at least one additional UVB filter, at least one micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

Preferably, the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof is mixed with at least one dermatologically acceptable emulsifier, thickener, emollient, and/or water.

The present invention further relates to the use of a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof in the manufacture of a sunscreen or daily care composition having increased compatibility with skin microbiome, preferably human skin microbiome. It is to be understood that above embodiments of the use aspect of the sunscreen or daily care composition are also applicable for this aspect, in particular regarding the microorganisms, the at least one additional UVB filter, at least one micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

As mentioned above, the invention relates in a second aspect to a sunscreen or daily care composition comprising
0.1 to 10 wt.-%, preferably 0.5 to 5 wt.-%, of hexyl 2-[4-(diethylamino)-2-hydroxybenzoyl]benzoate (INCI diethylamino hydroxybenzoyl hexyl benzoate),
0.1 to 8 wt.-%, preferably 0.5 to 4 wt.-%, of 2-Phenyl-3H-benzimidazole-5-sulfonic acid (INCI Phenylbenzimidazole Sulfonic Acid),
0.1 to 5 wt.-%, preferably 0.5 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 5 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0 to 10 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 15 wt.-%, preferably 5 to 10 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), zinc oxide, and mixtures thereof.

In preferred embodiment, 2,2′-methylene bis[6-(2H-benzotriazol-2-yl)-4-(1,1,3,3-tetramethylbutyl)phenol](INCI methylene bis-benzotriazolyl tetramethylbutylphenol) is not dispersed in (3R,4S,5S,6R)-2-(Decyloxy)-6-(hydroxymethyl)tetrahydro-2H-Pyran-3,4,5-triol (INCI decyl glucoside).

In a preferred embodiment, the sunscreen or daily care composition comprises at least one additional organic micronized UV filter, preferably selected from the group consisting of 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine). In this connection it is to be understood that the at least one additional organic micronized UV filter can be any known in the art organic micronized UV filter other than 2,2′-methylene bis[6-(2H-benzotriazol-2-yl)-4-(1,1,3,3-tetramethylbutyl)phenol](INCI methylene bis-benzotriazolyl tetramethylbutylphenol).

In preferred embodiment the at least one additional organic micronized UV filter is not dispersed in (3R,4S,5S,6R)-2-(Decyloxy)-6-(hydroxymethyl)tetrahydro-2H-Pyran-3,4,5-triol (INCI decyl glucoside).

In a preferred embodiment, the at least one additional organic micronized UV filter is dispersed in polyglycerol monoalkyl ester.

In a preferred embodiment, the 2,2′-methylene bis[6-(2Hbenzotriazol-2-yl)-4-(1,1,3,3-tetramethylbutyl)phenol](INCI Methylene bis-benzotriazolyl tetramethylbutylphenol) and the 2,4,6-tris(biphenyl-4-yl)-1,3,5-triazine (INCI Tris-Biphenyl triazine) are not dispersed in (3R,4S,5S,6R)-2-(Decyloxy)-6-(hydroxymethyl)tetrahydro-2H-Pyran-3,4,5-triol (INCI decyl glucoside).

In a preferred embodiment, the 2,2′-methylene bis[6-(2Hbenzotriazol-2-yl)-4-(1,1,3,3-tetramethylbutyl)phenol](INCI Methylene bis-benzotriazolyl tetramethylbutylphenol) and the 2,4,6-tris(biphenyl-4-yl)-1,3,5-triazine (INCI Tris-Biphenyl triazine) are dispersed in polyglycerol monoalkyl ester.

Preferably, the polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and mixtures thereof, more preferably wherein the at least one polyglycerol monoalkyl ester is decaglyceryl monolaurate (INCI Polyglyceryl-10 Laurate).

In a preferred embodiment, the sunscreen or daily care composition comprises 2,2′-methylene bis[6-(2Hbenzotriazol-2-yl)-4-(1,1,3,3-tetramethylbutyl)phenol](INCI Methylene bis-benzotriazolyl tetramethylbutylphenol) dispersed in polyglycerol monoalkyl ester and/or the 2,4,6-tris(biphenyl-4-yl)-1,3,5-triazine (INCI Tris-Biphenyl triazine) dispersed in polyglycerol monoalkyl ester.

It is to be understood that the above embodiments of the use of the sunscreen or daily care composition are also applicable for the second aspect, in particular regarding the at least one additional UVB filter, at least one micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition. Further preferred embodiments of the sunscreen or daily care composition of the present invention are disclosed in the following.

Preferably, the inventive sunscreen or daily care composition is microbiome friendly and therefore suitable for any skin type. The inventive sunscreen or daily care composition is particularly suitable for sensitive skin.

The sunscreen or daily care composition can be administered on any skin type, even on sensitive skin.

In a particular preferred embodiment, the sunscreen or daily care composition comprises
0.1 to 6 wt.-%, preferably 0.4 to 5 wt.-%, of hexyl 2-[4-(diethylamino)-2-hydroxybenzoyl]benzoate (INCI diethylamino hydroxybenzoyl hexyl benzoate),
0.1 to 6 wt.-%, preferably 0.4 to 4 wt.-%, of 2-Phenyl-3H-benzimidazole-5-sulfonic acid (INCI Phenylbenzimidazole Sulfonic Acid),
0.1 to 4 wt.-%, preferably 0.4 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 4 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0 to 6 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 8 wt.-%, preferably 5 to 8 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), homosalate, zinc oxide, and mixtures thereof.

In a preferred embodiment, the sunscreen or daily care composition comprises Sodium Stearoyl Glutamate in an amount of 0.1 to 2 wt.-%, preferably of 1 to 2 wt.-% or 0.3 to 1 wt.-%, and in particular of 0.2 to 1 wt.-%, based on the total weight of the sunscreen or daily care composition.

In a preferred embodiment, the sunscreen or daily care composition comprises a monoester of stearic acid with glycerin (e.g. glyceryl stearate SE) in an amount of 1 to 4 wt.-%, preferably 1.5 to 3 wt.-%, based on the total weight of the sunscreen or daily care composition.

In a preferred embodiment, the sunscreen or daily care composition does not contain zinc derivative compounds (having the meaning as denotes above).

In a preferred embodiment, the sunscreen or daily care composition does not contain a zinc salt. In a particular preferred embodiment, the sunscreen or daily care composition does not contain a zinc salt of pyrrolidone carboxylic acid.

In a preferred embodiment, the sunscreen or daily care composition does not contain parabens.

In a preferred embodiment, the sunscreen or daily care composition does not contain 3,3,5-Trimethylcyclohexyl 2-hydroxybenzoate (INCI homosalate).

In a preferred embodiment, the sunscreen or daily care composition does not contain PVP (polyvinylpyrrolidone) polymers and VP (vinylpyrrolidone) derivatives such as vinylpyrrolidone/hexadecene copolymer, VP/Eicosene Copolymer, Triacontanyl PVP.

In a preferred embodiment, the sunscreen or daily care composition does not contain polymers based on acrylic acid such as for example not limited to compounds with the INCI carbomers, C10/30 alkyl acrylate crosspolymer.

In a preferred embodiment, the sunscreen or daily care composition does not contain ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), and zinc oxide.

In a preferred embodiment, the sunscreen or daily care composition comprises
0.1 to 10 wt.-%, preferably 0.5 to 5 wt.-%, of hexyl 2-[4-(diethylamino)-2-hydroxybenzoyl]benzoate (INCI diethylamino hydroxybenzoyl hexyl benzoate),
0.1 to 8 wt.-%, preferably 0.5 to 4 wt.-%, of 2-Phenyl-3H-benzimidazole-5-sulfonic acid (INCI Phenylbenzimidazole Sulfonic Acid),
0.1 to 5 wt.-%, preferably 0.5 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 5 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0.1 to 10 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 15 wt.-%, preferably 5 to 10 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), zinc oxide, and mixtures thereof.

In a particular preferred embodiment, the sunscreen or daily care composition comprises
0.1 to 6 wt.-%, preferably 0.4 to 5 wt.-%, of hexyl 2-[4-(diethylamino)-2-hydroxybenzoyl]benzoate (INCI diethylamino hydroxybenzoyl hexyl benzoate),
0.1 to 6 wt.-%, preferably 0.4 to 4 wt.-%, of 2-Phenyl-3H-benzimidazole-5-sulfonic acid (INCI Phenylbenzimidazole Sulfonic Acid),
0.1 to 4 wt.-%, preferably 0.4 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 4 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0.1 to 6 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol),
3 to 8 wt.-%, preferably 5 to 8 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), 3,3,5-Trimethylcyclohexyl 2-hydroxybenzoate (INCI homosalate), zinc oxide, and mixtures thereof.

In a preferred embodiment, the 2,2′-methylene bis[6-(2Hbenzotriazol-2-yl)-4-(1,1,3,3-tetramethylbutyl)phenol](INCI Methylene bis-benzotriazolyl tetramethylbutylphenol) is not dispersed in (3R,4S,5S,6R)-2-(Decyloxy)-6-(hydroxymethyl)tetrahydro-2H-Pyran-3,4,5-triol (INCI decyl glucoside).

Preferably, the 2,2′-methylene bis[6-(2Hbenzotriazol-2-yl)-4-(1,1,3,3-tetramethylbutyl)phenol](INCI Methylene bis-benzotriazolyl tetramethylbutylphenol) is dispersed in polyglycerol monoalkyl ester.

Preferably, the polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and mixtures thereof, more preferably wherein the at least one polyglycerol monoalkyl ester is decaglyceryl monolaurate (INCI Polyglyceryl-10 Laurate).

In a particular preferred embodiment, the sunscreen or daily care composition comprises
0.1 to 6 wt.-%, preferably 0.4 to 5 wt.-%, of hexyl 2-[4-(diethylamino)-2-hydroxybenzoyl]benzoate (INCI diethylamino hydroxybenzoyl hexyl benzoate),
0.1 to 6 wt.-%, preferably 0.4 to 4 wt.-%, of 2-Phenyl-3H-benzimidazole-5-sulfonic acid (INCI Phenylbenzimidazole Sulfonic Acid),
0.1 to 4 wt.-%, preferably 0.4 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 4 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0.1 to 6 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 8 wt.-%, preferably 5 to 8 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p-Methoxycinnamate), ethylhexyl-2-cyano-3,3-diphenyl-acrylate (INCI octocrylene), zinc oxide, and mixtures thereof and wherein the 2,2′-methylene bis[6-(2Hbenzotriazol-2-yl)-4-(1,1,3,3-tetramethylbutyl)phenol](INCI Methylene bis-benzotriazolyl tetramethylbutylphenol) is not dispersed in (3R,4S,5S,6R)-2-(Decyloxy)-6-(hydroxymethyl)tetrahydro-2H-Pyran-3,4,5-triol (INCI decyl glucoside).

In a preferred embodiment, the sunscreen or daily care composition further comprises C₂-C₁₂-alkandiol, preferably a 1,2-C₂-C₁₂-alkandiol or a 1,3-C₂-C₁₂-alkandiol, more preferably a 1,2-C₂-C₁₀-alkandiol or a 1,3-C₂-C₁₀-alkandiol, and in particular 1,2-C₂-C₁₀-alkandiol or 1,2-C₂-C₈-alkandiol. Preferably, the sunscreen or daily care composition comprises the C₂-C₁₂-alkandiol in an amount of from 0.5 to 10 wt.-%, more preferably from 1 to 8 wt.-%, and based on the total weight of the sunscreen or daily care composition.

In a preferred embodiment, the sunscreen or daily care composition further comprises glycerol. Preferably, the sunscreen or daily care composition comprises the glycerol in an amount of from 1 to 10 wt.-%, more preferably from 2 to 8 wt.-% or from 4 to 10 wt.-%, and in particular from 3 to 7 wt.-% or from 4 to 8 wt.-%, based on the total weight of the sunscreen or daily care composition.

In a preferred embodiment, the sunscreen or daily care composition comprises DHHB and glycerol in a weight ratio of 3:1 to 1:10, preferably of 2:1 to 1:9, more preferably of 1:1 to 1:9, and in particular of 1:2 to 1:7.

In a preferred embodiment, the sunscreen or daily care has a SPF value of 6 to 50+(according to the EC recommendation). The SPF and UVA-PF can exemplarily be determined by simulation calculation using BASF sunscreen simulator (https://sunscreensimulator.basf.com/) or in vitro. A suitable in vitro method may be performed as follows:

SPF in vitro and UVA-PF in vitro was determined based on ISO 24443, which is based on the assessment of UV-transmittance through a thin film of sunscreen sample spread on roughened substrate—SB6 PMMA-plates (4-5 μm roughness, area 4.8×4.8 cm) from Helioscreen, Paris. The sunscreen formulation to be tested was applied on the PMMA plate with an amount of 1.2 mg/cm². Three plates and 5 measurements per plate were done per formulation. The formulation was distributed manually as equal as possible on the plate. The plates applied with formulation were stored 30 minutes at room temperature and in a dark place prior the measurement.

### Measurements:

### Step 1

In vitro transmission measurement were performed from 290-400 nm with 1 nm steps prior to any UV irradiation with the Labsphere UV Transmittance Analyzer UV 2000S. The initial UV transmission spectrum was acquired and the SPF in vitro values were calculated before irradiation (mean SPF of all 15 values (from the three plates and 5 measurements per plate))

### Step 2

A mathematical adjustment of the initial UV spectrum was performed with a coefficient “c” as described in ISO 24443 and the UVA-PF in vitro was calculated before irradiation.

### Step 3

The UV irradiation with Atlas Suntest CPS+ Solar simulator of the formulation was calculated with the UV Dose D as described in ISO 24443.

### Step 4

In vitro transmission measurement after UV exposure and acquisition of the UV transmission spectrum after irradiation were performed. The in vitro SPF value after irradiation was determined. (average of 15 values from the three plates and 5 measurements per plate)

### Step 5

A mathematical adjustment of the UV spectrum after irradiation was performed with the same coefficient “c” (determined step 2). The UVA-PF in vitro was calculated after UV exposure.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. The sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions or in the form of a sprayable product in aerosol packaging or not.

As mentioned above, the invention relates in a third aspect to the use of a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof for producing a sunscreen or daily care composition, which is compatible with the skin microbiome.

“Being compatible with the skin microbiome” denotes that a sunscreen or daily care composition (e.g. comprising a DHHB-containing composition (A)) at least has the same effect on the skin microbiome when compared to the exact same sunscreen or daily care composition, which merely does not comprise DHHB. This can be exemplarily determined by the above-outlined method detecting the skin microbiome microorganisms, preferably Staphylococcus epidermidis, population over e.g. 24 hours. Preferably, the sunscreen or daily care composition according to the present invention even provides an increase of the compatibility with the skin microbiome. The meaning of “increasing the compatibility of the sunscreen or daily composition with skin microbiome” is already outlined above in more detail.

It is to be understood that the above embodiments of the use of the sunscreen or daily care composition are also applicable for the third aspect, in particular regarding the microorganisms, the at least one additional UVB filter, at least one organic micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition. Further preferred embodiments of the sunscreen or daily care composition of the present invention are disclosed in the following.

In a preferred embodiment, the composition (A) comprises hexyl 2-[4-(diethylamino)-2-hydroxybenzoyl]benzoate (INCI diethylamino hydroxybenzoyl hexyl benzoate).

In a preferred embodiment, the composition (A) comprises a UV filter selected from the group consisting of hexyl 2-[4-(diethylamino)-2-hydroxybenzoyl]benzoate (INCI diethylamino hydroxybenzoyl hexyl benzoate), Terephthalylidene Dicamphor Sulfonic Acid (TDSA), drometrizole trisiloxane (DTS), and mixtures thereof.

In a preferred embodiment, the sunscreen or daily care composition comprises hexyl 2-[4-(diethylamino)-2-hydroxybenzoyl]benzoate (INCI diethylamino hydroxybenzoyl hexyl benzoate and further 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or 2-Phenyl-3H-benzimidazole-5-sulfonic acid (INCI Phenylbenzimidazole Sulfonic Acid).

In a preferred embodiment, the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10 wt.-%, preferably from 0.2 to 9 wt.-%, more preferably from 0.3 to 8 wt.-%, still more preferably from 0.4 to 6 wt.-%, and in particular from 0.5 to 5 wt.-%, based on the total weight of the sunscreen or daily care composition.

In a preferred embodiment, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 10 wt.-%, preferably from 0.3 to 6 wt.-%, and in particular from 0.5 to 4 wt. %, based on the total weight of the sunscreen or daily care composition.

In a preferred embodiment, the sunscreen or daily care composition comprises 2-Phenyl-3H-benzimidazole-5-sulfonic acid (INCI Phenylbenzimidazole Sulfonic Acid) in an amount of from 0.1 to 8 wt.-%, preferably from 0.3 to 6 wt.-%, and in particular from 0.5 to 4 wt.-%, based on the total weight of the sunscreen or daily care composition.

In a preferred embodiment, the composition (A) comprises hexyl 2-[4-(diethylamino)-2 hydroxybenzoyl]benzoate (INCI diethylamino hydroxybenzoyl hexyl benzoate) in an amount of from 70 to 100 wt.-%, preferably from 80 to 100 wt.-%, more preferably from 90 to 100 wt.-%, and in particular from 95 to 100 wt.-%, based on the total weight of the composition (A). In a particular embodiment the composition (A) consists of hexyl 2-[4-(diethylamino)-2 hydroxybenzoyl]benzoate (INCI diethylamino hydroxybenzoyl hexyl benzoate).

In a preferred embodiment, the composition (A) comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 70 to 100 wt.-%, preferably from 80 to 100 wt.-%, more preferably from 90 to 100 wt.-%, and in particular from 95 to 100 wt.-%, based on the total weight of the composition (A). In a particular embodiment the composition (A) consists of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine).

In a preferred embodiment, the composition (A) comprises Phenyl-3H-benzimidazole-5-sulfonic acid (INCI Phenylbenzimidazole Sulfonic Acid) in an amount of from 70 to 100 wt.-%, preferably from 80 to 100 wt.-%, more preferably from 90 to 100 wt.-%, and in particular from 95 to 100 wt.-%, based on the total weight of the composition (A). In a particular embodiment the composition (A) consists of Phenyl-3H-benzimidazole-5-sulfonic acid (INCI Phenylbenzimidazole Sulfonic Acid).

In a preferred embodiment, the composition (A) comprises Terephthalylidene Dicamphor Sulfonic Acid (TDSA) in an amount of from 70 to 100 wt.-%, preferably from 80 to 100 wt.-%, more preferably from 90 to 100 wt.-%, and in particular from 95 to 100 wt.-%, based on the total weight of the composition (A). In a particular embodiment the composition (A) consists of Terephthalylidene Dicamphor Sulfonic Acid (TDSA).

In a preferred embodiment, the composition (A) comprises drometrizole trisiloxane (DTS) in an amount of from 70 to 100 wt.-%, preferably from 80 to 100 wt.-%, more preferably from 90 to 100 wt.-%, and in particular from 95 to 100 wt.-%, based on the total weight of the composition (A). In a particular embodiment the composition (A) consists of drometrizole trisiloxane (DTS).

In a preferred embodiment, the sunscreen or daily care composition is produced by adding the composition (A) into an oil phase. Preferably, the composition (A), preferably comprising hexyl 2-[4-(diethylamino)-2 hydroxybenzoyl]benzoate (INCI diethylamino hydroxybenzoyl hexyl benzoate), is added under stirring. The oil phase may comprise any suitable additional UV filter known in the art. Particular suitable UV filters, which can be present in the oil phase are 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 4,4′,4″-(1,3,5-triazin-2,4,6-triyltriimino)tris-benzoic acid-tris(2-ethylhexyl)ester (INCI ethylhexyl triazone), and mixtures thereof. Preferably, the composition comprising composition (A), preferably comprising hexyl 2-[4-(diethylamino)-2 hydroxybenzoyl]benzoate (INCI diethylamino hydroxybenzoyl hexyl benzoate), and the oil phase is blended with at least one aqueous phase. In the aqueous phase, another suitable additional UV filter can be present. A particular suitable UV filter, which can be present in the aqueous phase is 2-Phenyl-3H-benzimidazole-5-sulfonic acid (INCI Phenylbenzimidazole Sulfonic Acid), Terephthalylidene Dicamphor Sulfonic Acid (TDSA).

In a preferred embodiment, the sunscreen or daily care composition is produced by adding the composition (A) into an oil phase. Preferably, the composition (A), preferably comprising drometrizole trisiloxane is added under stirring. The oil phase may comprise any suitable additional UV filter known in the art. Particular suitable UV filters, which can be present in the oil phase are 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 4,4′,4″-(1,3,5-triazin-2,4,6-triyltriimino)tris-benzoic acid-tris(2-ethylhexyl)ester (INCI ethylhexyl triazone), and mixtures thereof. Preferably, the composition comprising composition (A), preferably comprising drometrizole trisiloxane, and the oil phase is blended with at least one aqueous phase. In the aqueous phase, another suitable additional UV filter can be present. A particular suitable UV filter, which can be present in the aqueous phase are 2-Phenyl-3H-benzimidazole-5-sulfonic acid (INCI Phenylbenzimidazole Sulfonic Acid), Terephthalylidene Dicamphor Sulfonic Acid (TDSA).

In a preferred embodiment, the sunscreen or daily care composition is produced by adding the composition (A) into an aqueous phase. Preferably, the composition (A), preferably comprising 2-Phenyl-3H-benzimidazole-5-sulfonic acid (INCI Phenylbenzimidazole Sulfonic Acid), is added under stirring.

In a preferred embodiment, the sunscreen or daily care composition is produced by adding the composition (A) into an aqueous phase. Preferably, the composition (A), preferably comprising Terephthalylidene Dicamphor Sulfonic Acid (TDSA), is added under stirring. Preferably, the composition comprising composition (A), preferably comprising Terephthalylidene Dicamphor Sulfonic Acid (TDSA) is blended with at least one lipophilic phase. In the lipophilic phase, another suitable additional UV filter can be present. A particular suitable UV filter, which can be present in the lipophilic phase are hexyl 2-[4-(diethylamino)-2 hydroxybenzoyl]benzoate (INCI diethylamino hydroxybenzoyl hexyl benzoate), 4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 4,4′,4″-(1,3,5-triazin-2,4,6-triyltriimino)tris-benzoic acid-tris(2-ethylhexyl)ester (INCI ethylhexyl triazone).

The sunscreen or daily care composition comprises typical sunscreen or daily care composition ingredients (e.g. emulsifier, thickener, emollient, sensory enhancers, an adjuvants, a preservatives, and/or a perfumes). Preferably, the sunscreen or daily care composition comprises glycerol. Preferably, the sunscreen or daily care composition comprises the glycerol in an amount of from 1 to 10 wt.-%, more preferably from 2 to 8 wt.-% or from 4 to 10 wt.-%, and in particular from 5 to 8 wt.-%, based on the total weight of the sunscreen or daily care composition.

In a preferred embodiment, the sunscreen or daily care has a SPF value of 6 to 50+(according to the EC recommendation), the SPF is determined in vitro, more preferably according to the below outlined method (according to the examples).

The invention further relates to a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS) and mixtures thereof, for use in a method of producing a sunscreen or daily care composition, which is compatible with the skin microbiome, preferably human skin microbiome. It is to be understood that the above embodiments of the use aspect of the sunscreen or daily care composition and of the use aspect of composition (A) are also applicable for this aspect, in particular regarding the microorganisms, the at least one additional UVB filter, at least one organic micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

The present invention further relates to a method of producing a sunscreen or daily care composition, which is compatible with the skin microbiome, preferably human skin microbiome, the method comprising the step of mixing a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS) and mixtures thereof, with at least one other dermatologically acceptable ingredient.

It is to be understood that above embodiments of the use aspect of the sunscreen or daily care composition and of the use aspect of composition (A) are also applicable for this aspect, in particular regarding the microorganisms, the at least one additional UVB filter, at least one organic micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

Preferably, the composition (A) is mixed with at least one dermatologically acceptable emulsifier, thickener, and/or emollient.

The present invention further relates to the use a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, in the manufacture of a sunscreen or daily care composition having increased compatibility with the skin microbiome, preferably human skin microbiome. It is to be understood that above embodiments of the use aspect of the sunscreen or daily care composition and of the use aspect of composition (A) are also applicable for this aspect, in particular regarding the microorganisms, the at least one additional UVB filter, at least one organic micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

The invention further relates to the following items:
1. A sunscreen or daily care composition comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), and mixtures thereof, for use in a method of increasing the compatibility of the sunscreen or daily composition with skin microbiome.
2. The sunscreen or daily care composition according to item 1, comprising hexyl 2-[4-(diethylamino)-2-hydroxybenzoyl]benzoate (INCI diethylamino hydroxybenzoyl hexyl benzoate) and optionally further comprising 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or 2-Phenyl-3H-benzimidazole-5-sulfonic acid (INCI Phenylbenzimidazole Sulfonic Acid).
3. The sunscreen or daily care composition according to item 1 or 2, wherein the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), and mixtures thereof, increases the compatibility of the sunscreen or daily care composition with the population of bacteria selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium tuberculostearicum, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., and mixtures thereof, preferably Staphylococcus epidermidis, on the skin and/or
increases the compatibility of the sunscreen or daily care composition with the population of fungi selected from the group consisting of Malassezia furfur; Malassezia restricta, Malassezia globosa, Candida spp., and mixtures thereof on the skin.
4. The sunscreen or daily care composition according to any one of items 1 to 3, further comprising at least one additional UVB filter, preferably selected from the group consisting of 4,4′,4″-(1,3,5-triazin-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), dimethicone diethyl benzylmalonate (INCI polysilixone-15), and mixtures thereof, and in particular selected from the group consisting of 4,4′,4″-(1,3,5-triazin-2,4,6-triyltriimino)tris-benzoic acid-tris(2-ethylhexyl)ester (INCI ethylhexyl triazone or EHT), 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), and mixtures thereof.
5. The sunscreen or daily care composition according to any one of items 1 to 4, further comprising at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine) preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine).
6. The sunscreen or daily care composition according to item 5, wherein the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside) and/or wherein the at least one organic micronized UV filter is dispersed in polyglycerol monoalkyl ester.
7. The sunscreen or daily care composition according to any one of items 1 to 6, further comprising at least one additional UVA filter, preferably selected from the group consisting of disodium phenyl dibenzimidazole tetrasulfonate, 1-(4-(1,1-dimethylethyl)phenyl)-3-(4-methoxyphenyl)propane-1,3-dione (INCI Butyl Methoxydibenzoylmethane), and mixtures thereof, in particular 1-(4-(1,1-dimethylethyl)phenyl)-3-(4-methoxyphenyl)propane-1,3-dione (INCI Butyl Methoxydibenzoylmethane).
8. The sunscreen or daily care composition according to any one of items 1 to 7, wherein the sunscreen or daily care composition does not contain zinc oxide (ZnO).
9. The sunscreen or daily care composition according to any one of items 1 to 8, comprising hexyl 2-[4-(diethylamino)-2 hydroxybenzoyl]benzoate (INCI diethylamino hydroxybenzoyl hexyl benzoate) in an amount of from 0.1 to 10 wt.-%, preferably from 0.5 to 8 wt.-%, and in particular from 0.5 to 5 wt.-%, based on the total weight of the sunscreen or daily care composition.
10. The sunscreen or daily care composition according to any one of items 1 to 9, comprising 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7 wt.-%, preferably from 0.5 to 4 wt. %, based on the total weight of the sunscreen or daily care composition and/or 2-Phenyl-3H-benzimidazole-5-sulfonic acid (INCI Phenylbenzimidazole Sulfonic Acid) in an amount of from 0.1 to 6 wt.-%, preferably from 0.5 to 4 wt.-%, based on the total weight of the sunscreen or daily care composition.
11. The sunscreen or daily care composition according to any one of items 1 to 10, comprising at least one organic micronized UV filter in an amount of from 0.1 to 10 wt. %, preferably from 0.5 to 5 wt.-%, based on the total weight of the sunscreen or daily care composition.
12. The sunscreen or daily care composition according to any one of items 1 to 11, comprising 4,4′,4″-(1,3,5-triazin-2,4,6-triyltriimino)tris-benzoic acid-tris(2-ethylhexyl)ester (INCI ethylhexyl triazone) in an amount of from 0.1 to 5 wt.-%, preferably from 0.5 to 4 wt.-%, based on the total weight of the sunscreen or daily care composition.
13. The sunscreen or daily care composition according to any one of items 1 to 12, further comprising a dermatologically acceptable emulsifier, thickener, and/or emollient.
14. The sunscreen or daily care composition according to any one of items 1 to 13, wherein the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate) and/or 3-Methylbutyl (2E)-3-(4-methoxyphenyl)prop-2-enoate (INCI Isoamyl p-Methoxycinnamate).
15. A sunscreen or daily care composition comprising
0.1 to 10 wt.-%, preferably 0.5 to 5 wt.-%, of hexyl 2-[4-(diethylamino)-2-hydroxybenzoyl]benzoate (INCI diethylamino hydroxybenzoyl hexyl benzoate),
0.1 to 8 wt.-%, preferably 0.5 to 4 wt.-%, of 2-Phenyl-3H-benzimidazole-5-sulfonic acid (INCI Phenylbenzimidazole Sulfonic Acid),
0.1 to 5 wt.-%, preferably 0.5 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 5 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0 to 10 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 15 wt.-%, preferably 5 to 10 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p_Methoxycinnamate), ethylhexyl-2-cyano-3,3-diphenyl-acrylate (INCI octocrylene), zinc oxide, and mixtures thereof.
16. A composition (A) comprising hexyl 2-[4-(diethylamino)-2-hydroxybenzoyl]benzoate (INCI diethylamino hydroxybenzoyl hexyl benzoate) for use in a method of producing a sunscreen or daily care composition, which is compatible with the skin microbiome.
17. The composition (A) according to item 16, wherein the sunscreen or daily care composition further comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or 2-Phenyl-3H-benzimidazole-5-sulfonic acid (INCI Phenylbenzimidazole Sulfonic Acid).

### Examples

The present invention is further illustrated by the following examples.

### Evaluation of SPF

The SPF values were calculated according to the BASF sunscreen simulator (https://sunscreensimulator.basf.com/).

### Determining the Antibacterial Activity (AA) to Evaluate the Microbial Compatibility Bacteria:

Staphylococcus epidermidis, Micrococcus luteus, Corynebacterium jeikeium, and Malassezia furfur (typical representatives of human skin flora) were used.

### Inoculum:

Second subculture of S. epidermidis and Micrococcus luteus on tryptic soy agar was suspended in tryptic soy broth and incubated overnight (overnight culture, 24 h at 36° C.).

Second subculture of Corynebacterium jeikeium on tryptic soy agar was suspended in tryptic soy broth containing 1.0% polysorbate 80 and incubated overnight (24 h) at 36° C.

Second subculture of Malassezia furfur on Medium 472 agar was suspended in medium 472 broth and incubated for 6 days at 30° C. (medium 472=472. PITYROSPORUM MEDIUM ex DSMZ without addition of agar).

### Test Design:

Each tested formulation was contaminated (TO) with the microbial suspension (e.g. dilution of overnight culture of S. epidermidis in sodium chloride)

Staphylococcus epidermidis & Micrococcus luteus:

At 2 h, 4 h, and/or 24 h contact, each sample was spread out on the surface of tryptone soy agar plates & incubated for 2 days at 36° C. before microbial enumeration

Corynebacterium jeikeium:

At 2 h, 4 h, and/or 24 h contact, each sample was spread out on the surface of tryptone soy agar plates containing 1.0% polysorbate 80 and incubated for 2 days at 36° C. before microbial enumeration

Malassezia furfur:

At 2 h, 4 h, and/or 24 h contact, each sample was spread out on the surface of medium 472 agar plates & incubated for 6 days at 30° C. before microbial enumeration (medium 472=472. PITYROSPORUM MEDIUM ex DSMZ)

### Evaluation:

Counting of the total number of colony forming units, cfu/mL, on each agar plate for all test conditions

### Formulations:

1% active amount of tested UV filters were added (formulations 1 to 14 and 16 to 18, Tables 2a to 2d)
Placebo emulsion without UV filters (formulation 15, Tables 2a to 2c)
Combination of UV filters (formulations 19 to 21, Table 3)
Combination of UV filters (formulations 22 to 24, Table 4)
Combination of UV filers (formulations 25 to 27, Table 5)
Combination of UV filters (formulations 28 to 30, Table 6)
Combination of UV filters (formulations 31 to 34, Table 7)

The antibacterial activity (AA) to evaluate the microbial compatibility as denoted in Tables 2a to 2d was categorized into the following ten groups.

Groups 2_1 to 2_5 (Value after 2 Hours of Contact):
Group 2_1: Number in cfu/mL of tested microorganism is in a range of 10⁵ to less than 10⁶
Group 2_2: Number in cfu/mL of tested microorganism is in a range of 10⁴ to less than 10⁵
Group 2_3: Number in cfu/mL of tested microorganism is in a range of 10³ to less than 10⁴
Group 2_4: Number in cfu/mL) of tested microorganism is in a range of 10² to less than 10³
Group 2_5: Number in cfu/mL of tested microorganism is in a range of less than 10¹
   Groups 4_1 to 4_5 (Value after 4 Hours of Contact):
Group 4_1: Number in cfu/mL of tested microorganism is in a range of 10⁵ to less than 10⁶
Group 4_2: Number in cfu/mL of tested microorganism is in a range of 10⁴ to less than 10⁵
Group 4_3: Number in cfu/mL of tested microorganism is in a range of 10³ to less than 10⁴
Group 4_4: Number in cfu/mL) of tested microorganism is in a range of 10² to less than 10³
Group 4_5: Number in cfu/mL of tested microorganism is in a range of less than 10¹
   Groups 24_1 to 24_5 (Value after 24 Hours of Contact):
Group 24_1: Number in cfu/mL of tested microorganism is in a range of 10⁵ to less than 10⁶
Group 24_2: Number in cfu/mL of tested microorganism is in a range of 10⁴ to less than 10⁵
Group 24_3: Number in cfu/mL of tested microorganism is in a range of 10³ to less than 10⁴
Group 24_4: Number in cfu/mL of tested microorganism is in a range of 10² to less than 10³
Group 24_5: Number in cfu/mL of tested microorganism is in a range of less than 10¹
   Formulations 1 to 18 were Manufactured as Follows:

Phase A and part B1 were heated to 80° C. Part B2 was added under stirring, when homogeneous part B3 was added under high speed homogenizer (Ultra Turrax type device). Phase A was added into Phase B under stirring and homogenized (with an Ultra Turrax type device). The mixture was cooled down under stirring, adjusted to pH of approx. 6.5 with a sodium hydroxide solution.

TABLE 1
Ingredient of formulations 1 to 18

| | Trade name | INCI | Amount [wt.-%] |
|---|---|---|---|
| A | Dehymuls PGPH | Polyglyceryl-2 | 1.00 |
| | | Dipolyhydroxystearate | |
| | Cetiol CC | Dicaprylyl Carbonate | 3.00 |
| | Cetiol 4 All | Dipropylheptyl Carbonate | 3.00 |
| | Cetiol RLF | Caprylyl Caprylate/Caprate | 10.00 |
| | UV Filter, | See Table 2 | 1.00 |
| | if present | | |
| B1 | | Aqua | Ad 100% |
| | | Glycerol | 5.00 |
| | 1,3-Butanediol | Butylene Glycol | 10.00 |
| B2 | Rheocare XGN | Xanthan Gum | 2.00 |
| B3 | Plantapon LC 7 | Laureth-7 Citrate | 0.5 |

The antibacterial activity to evaluate the microbial compatibility of formulations 1 to 18 was tested. As can be seen from Table 2a, the respectively used UV filters have an impact on the Staphylococcus epidermidis population.

TABLE 2a
Formulations comprising one or no UV filter; AA denotes the antibacterial activity
to evaluate the microbial compatibility with Staphylococcus epidermidis in
cfu/mL after 4 or 24 hours contact of the tested formulations 1 to 16, wherein
formulations 1 to 14 and 16 to 18 comprises 1 wt.-% of the respective UV filter
and formulation 15 does not comprise a UV filter (placebo formulation).

| | | | | AA, | AA, |
|---|---|---|---|---|---|
| No | Tested compound | INCI | Abbreviation | 4 h | 24 h |
| 1 | hexyl 2-[4-(diethylamino)-2- | Diethylamino | DHHB | 4_1 | 24_2 |
| | hydroxybenzoyl]benzoate | Hydroxybenzoyl | | | |
| | | Hexyl Benzoate | | | |
| 2 | 2,4-Bis-{[4-(2-ethyl-hexyloxy)- | Bis-ethylhexyloxyphenol | BEMT | 4_1 | 24_2 |
| | 2-hydroxy]-phenyl}-6-(4- | Methoxyphenyl triazine | | | |
| | methoxyphenyl)-1,3,5-triazine | | | | |
| 3 | 2-Phenyl-3H-benzimidazole-5- | Phenylbenzimidazole | PBSA | 4_1 | 24_2 |
| | sulfonic acid | Sulfonic Acid | | | |
| 4 | 2,2′-Methylenebis[6-(2H-1,2,3- | Methylene | MBBT | 4_1 | 24_3 |
| | benzotriazol-2-yl)-4-(2,4,4- | Bis-Benzotriazolyl | | | |
| | trimethylpentan-2-yl)phenol]* | Tetramethylbutylphenol | | | |
| 5 | (RS)-2-Ethylhexyl (2E)-3-(4- | Ethylhexyl | EHMC | 4_1 | 24_3 |
| | methoxyphenyl)prop-2-enoate | Methoxycinnamate | | | |
| 6 | 2-Ethylhexyl 2-cyano-3,3- | Octocrylene | OCR | 4_1 | 24_3 |
| | diphenylprop-2-enoate | | | | |
| 7 | 2-ethylhexyl 2-hydroxybenzoate | Ethylhexyl Salicylate | EHS | 4_1 | 24_3 |
| 8 | of 2,2′-methylene bis[6- | Butyl Methoxydibenzoyl | BMDBM | 4_1 | 24_3 |
| | (2Hbenzotriazol-2-yl)-4-(1,1,3,3- | Methane | | | |
| | tetramethylbutyl)phenol] | | | | |
| 9 | Titanium Dioxide | | TiO2 | 4_2 | 24_3 |
| 10 | 4,4′,4″-(1,3,5-triazin-2,4,6- | Ethylhexyl Triazone | EHT | 4_1 | 24_4 |
| | triyltriimino)tris-benzoic acid- | | | | |
| | tris(2-ethylhexyl)ester | | | | |
| 11 | Bis(2-ethylhexyl) | Diethylhexyl Butamido | DBT | 4_2 | 24_4 |
| | 4,4′-[(6-{[4-(tert- | Triazone | | | |
| | butylcarbamoyl)phenyl]amino}- | | | | |
| | 1,3,5-triazine-2,4- | | | | |
| | diyl)bis(azanediyl)]dibenzoate | | | | |
| 12 | 2,2′-Methylenebis[6-(2H-1,2,3- | Methylene | MBBT | 4_2 | 24_5 |
| | benzotriazol-2-yl)-4-(2,4,4- | Bis-Benzotriazolyl | | | |
| | trimethylpentan-2-yl)phenol]** | Tetramethylbutylphenol | | | |
| 13 | 2,4,6-Tris(biphenyl-4-yl)- | Tris-biphenyl Triazine | TBPT | 4_2 | 24_5 |
| | 1,3,5-triazin* | | | | |
| 14 | Zinc oxide | | ZnO | 4_5 | 24_5 |
| 15 | — | — | — | 4_2 | 24_4 |
| 16 | 2,4,6-Tris(biphenyl-4-yl)- | Tris-biphenyl Triazine | TBPT | 4_1 | 24_2 |
| | 1,3,5-triazin** | | | | |
| 17 | 3,3′-(1,4- | Terephthalylidene | TDSA | 4_1 | 24_2 |
| | Phenylenedimethylene)Bis[7,7- | Dicamphor | | | |
| | Dimethyl-2-Oxo- | Sulfonic Acid | | | |
| | Bicyclo[2.2.1]Heptane-1- | | | | |
| | Methanesulfonic Acid] | | | | |
| 18 | 2-(2H-Benzotriazol-2-yl)-4- | Drometrizole trisiloxane | DTS | 4_1 | 24_1 |
| | methyl-6-[2-methyl-3-[1,3,3,3- | | | | |
| | tetramethyl-1-[(trimethylsilyl)oxy]- | | | | |
| | 1-disiloxanyl]propyl]phenol | | | | |

*dispersed with decyl glucoside
**dispersed with polyglyceryl-10 Laurate

Further, the antibacterial activity to evaluate the microbial compatibility of formulations 1 to 3 and 15 was tested. As can be seen from Tables 2b and 2c, the respectively used UV filters have a positive impact on the Micrococcus luteus and Corynebacterium jeikeium population.

TABLE 2b
Formulations comprising one or no UV filter; AA denotes the antibacterial activity to evaluate
the compatibility with Micrococcus luteus in cfu/mL after 4 or 24 hours contact of the tested
formulations 1 to 3, and 15, wherein formulations 1 to 3 comprises 1 wt.-% of the respective
UV filter and formulation 15 does not comprise a UV filter (placebo formulation).

| | | | | AA, | AA, |
|---|---|---|---|---|---|
| No | Tested compound | INCI | Abbreviation | 4 h | 24 h |
| 1 | hexyl 2-[4-(diethylamino)-2- | Diethylamino | DHHB | 4_1 | 24_1 |
| | hydroxybenzoyl]benzoate | Hydroxybenzoyl Hexyl | | | |
| | | Benzoate | | | |
| 2 | 2,4-Bis-{[4-(2-ethyl-hexyloxy)-2-hydroxy]- | Bis-ethylhexyloxyphenol | BEMT | 4_1 | 24_1 |
| | phenyl}-6-(4-methoxyphenyl)-1,3,5-triazine | Methoxyphenyl triazine | | | |
| 3 | 2-Phenyl-3H-benzimidazole-5-sulfonic acid | Phenylbenzimidazole | PBSA | 4_1 | 24_1 |
| | | Sulfonic Acid | | | |
| 15 | — | — | — | 4_3 | 24_3 |

TABLE 2c
Formulations comprising one or no UV filter; AA denotes the antibacterial activity to evaluate
the compatibility with Corynebacterium jeikeium in cfu/mL after 4 hours contact of the tested
formulations 1 to 3, and 15, wherein formulations 1 to 3 comprises 1 wt.-% of the respective
UV filter and formulation 15 does not comprise a UV filter (placebo formulation).

| | | | | AA, |
|---|---|---|---|---|
| No | Tested compound | INCI | Abbreviation | 4 h |
| 1 | hexyl 2-[4-(diethylamino)-2- | Diethylamino | DHHB | 4_3 |
| | hydroxybenzoyl]benzoate | Hydroxybenzoyl Hexyl | | |
| | | Benzoate | | |
| 2 | 2,4-Bis-{[4-(2-ethyl-hexyloxy)-2-hydroxy]- | Bis-ethylhexyloxyphenol | BEMT | 4_2 |
| | phenyl}-6-(4-methoxyphenyl)-1,3,5-triazine | Methoxyphenyl triazine | | |
| 3 | 2-Phenyl-3H-benzimidazole-5-sulfonic acid | Phenylbenzimidazole | PBSA | 4_2 |
| | | Sulfonic Acid | | |
| 15 | — | — | — | 4_4 |

TABLE 2d
Formulations comprising one UV filter; AA denotes the antibacterial activity to evaluate
the compatibility with Malassezia furfur in cfu/mL after 2 hours contact of the
tested formulations 1 to 3 comprising 1 wt.-% of the respective UV filter.

| | | | | AA, |
|---|---|---|---|---|
| No | Tested compound | INCI | Abbreviation | 2 h |
| 1 | hexyl 2-[4-(diethylamino)-2- | Diethylamino | DHHB | 2_3 |
| | hydroxybenzoyl]benzoate | Hydroxybenzoyl Hexyl | | |
| | | Benzoate | | |
| 2 | 2,4-Bis-{[4-(2-ethyl-hexyloxy)-2-hydroxy]- | Bis-ethylhexyloxyphenol | BEMT | 2_2 |
| | phenyl}-6-(4-methoxyphenyl)-1,3,5-triazine | Methoxyphenyl triazine | | |
| 3 | 2-Phenyl-3H-benzimidazole-5-sulfonic acid | Phenylbenzimidazole | PBSA | 2_2 |
| | | Sulfonic Acid | | |

For the formulations 19 to 21, Phase A and part B1 were heated to 80° C. Part B2 was added to part B under stirring, when homogeneous part B3 was added to B1/B2 under high speed homogenizer (Ultra Turrax type device). Phase A was added into Phase B under stirring and homogenized (with an Ultra Turrax type device). Phase C previously prepared was added to the emulsion, which was then cooled down under stirring and finally Phase 0 was added below 40° C. and the pH was adjusted to >7 with a sodium hydroxide solution.

TABLE 3
Blend formulations; AA denotes the antibacterial activity to evaluate
the microbial compatibility with Staphylococcus epidermidis in cfu/mL
after 4 or 24 hours contact of the tested formulations 19 to 21.

| | | | 19 | 20 | 21 |
|---|---|---|---|---|---|
| | | | Amount | Amount | Amount |
| | Trade name/tested compound | INCI | [%] | [%] | [%] |
| A | Dehymuls PGPH | Polyglyceryl-2 | 1.00 | 1.00 | 1.00 |
| | | Dipolyhydroxystearate | | | |
| | Cetiol CC | Dicaprylyl Carbonate | 3.00 | 3.00 | 3.00 |
| | Cetiol 4 All | Dipropylheptyl Carbonate | 3.00 | 3.00 | 3.00 |
| | Cetiol RLF | Caprylyl Caprylate/Caprate | 10.00 | 10.00 | 10.00 |
| | hexyl 2-[4-(diethylamino)-2- | diethylamino hydroxybenzoyl | 1.00 | 2.00 | — |
| | hydroxybenzoyl]benzoate | hexyl benzoate (DHHB) | | | |
| | 2,4-Bis-{[4-(2-ethyl-hexyloxy)- | bis-ethylhexyloxyphenol | 1.00 | 1.20 | — |
| | 2-hydroxy]-phenyl}-6-(4- | methoxyphenyl triazine | | | |
| | methoxyphenyl)-1,3,5-triazine | (BEMT) | | | |
| | 4,4′,4″-(1,3,5-triazin-2,4,6- | ethylhexyl triazone (EHT) | 1.0 | 1.00 | — |
| | triyltriimino)tris-benzoic acid- | | | | |
| | tris(2-ethylhexyl)ester | | | | |
| B1 | | Aqua | Ad | Ad | Ad |
| | | | 100% | 100% | 100% |
| | | Glycerol | 5.00 | 5.00 | 5.00 |
| | 1,3-Butanediol | Butylene Glycol | 10.00 | 10.00 | 10.00 |
| B2 | Rheocare XGN | Xanthan Gum | 2.00 | 2.00 | 2.00 |
| B3 | Plantapon LC 7 | Laureth-7 Citrate | 0.5 | 0.5 | 0.5 |
| C | Water | Aqua | 5.00 | 5.00 | — |
| | 2-Phenyl-3H-benzimidazole- | Phenylbenzimidazole | 1.00 | 1.00 | — |
| | 5-sulfonic acid | Sulfonic Acid (PBSA) | | | |
| | | Sodium hydroxide solution | Qs | Qs | — |
| | | | pH >7 | pH >7 | |
| D | 2,2′-Methylenebis[6-(2H-1,2,3- | Methylene Bis- | — | 0.50 | — |
| | benzotriazol-2-yl)-4-(2,4,4- | Benzotriazolyl | | | |
| | trimethylpentan-2-yl)phenol] | Tetramethylbutylphenol ** | | | |
| SPF | | | 10 | 15 | — |
| AA, | | | 4_2 | 4_2 | 4_2 |
| 4 h | | | | | |
| AA, | | | 24_3 | 24_3 | 24_4 |
| 24 h | | | | | |

** dispersed with polyglyceryl-10 Laurate

For the formulations 22 to 24, Phase A and part B1 were heated to 80° C. Part B2 was added under stirring into part B. Phase A was added into Phase B under stirring and homogenized (with an Ultra Turrax type device). Phase C previously prepared was added to the emulsion, which was cooled down under stirring and Phase 0 was finally added below 40° C. if present, and the pH was adjusted to >7 with a sodium hydroxide solution.

TABLE 4
Blend formulations; AA denotes the antibacterial activity to evaluate
the microbial compatibility with Staphylococcus epidermidis in cfu/mL
after 4 or 24 hours contact of the tested formulations 22 to 24.

| | | | 22 | 23 | 24 |
|---|---|---|---|---|---|
| | | | Amount | Amount | Amount |
| | Trade name/tested compound | INCI | [%] | [%] | [%] |
| A | | Glyceryl Stearate SE | 2.00 | 2.00 | 2.00 |
| | Eumulgin SG | Sodium Stearoyl | 0.20 | 0.20 | 0.20 |
| | | Glutamate | | | |
| | | C12-15 Alkyl benzoate | 10.00 | 10.00 | 10.00 |
| | Cetiol B | Dibutyl Adipate | 10.00 | 10.00 | 10.00 |
| | | Stearyl Alcohol | 1.50 | 1.50 | 1.50 |
| | | Hydrogenated Castor Oil | 2.00 | 2.00 | 2.00 |
| | Euxyl PE9010 | Phenoxyethanol (and) | 1.00 | 1.00 | 1.00 |
| | | Ethylhexylglycerin | | | |
| | hexyl 2-[4-(diethylamino)-2- | diethylamino hydroxybenzoyl | 2.00 | 2.50 | 3.00 |
| | hydroxybenzoyl]benzoate | hexyl benzoate (DHHB) | | | |
| | 2,4-Bis-{[4-(2-ethyl-hexyloxy)- | bis-ethylhexyloxyphenol | 2.00 | 3.00 | 3.00 |
| | 2-hydroxy]-phenyl}-6-(4- | methoxyphenyl triazine | | | |
| | methoxyphenyl)-1,3,5-triazine | (BEMT) | | | |
| | 4,4′,4″-(1,3,5-triazin-2,4,6- | ethylhexyl triazone (EHT) | 1.00 | 2.00 | 2.50 |
| | triyltriimino)tris-benzoic acid- | | | | |
| | tris(2-ethylhexyl)ester | | | | |
| | | Ethylhexyl salicylate | — | 2.00 | 3.00 |
| B1 | | Aqua | Ad | Ad | Ad |
| | | | 100% | 100% | 100% |
| | | Glycerol | 8.00 | 8.00 | 8.00 |
| B2 | Rheocare XGN | Xanthan Gum | 0.20 | 0.20 | 0.20 |
| C | Water | Aqua | 5.00 | 5.00 | 8.00 |
| | 2-Phenyl-3H-benzimidazole- | Phenylbenzimidazole | 1.00 | 2.00 | 3.00 |
| | 5-sulfonic acid | Sulfonic Acid (PBSA) | | | |
| | | Sodium hydroxide solution | Qs | Qs | Qs |
| | | | pH >7 | pH >7 | pH >7 |
| D | 2,2′-Methylenebis[6-(2H-1,2,3- | Methylene | — | — | 3.00 |
| | benzotriazol-2-yl)-4-(2,4,4- | Bis-Benzotriazolyl | | | |
| | trimethylpentan-2-yl)phenol] | Tetramethylbutylphenol ** | | | |
| SPF | | | 15 | 30 | 50 |
| AA, | | | 4_1 | 4_1 | 4_1 |
| 4 h | | | | | |
| AA, | | | 24_1 | 24_1 | 24_1 |
| 24 h | | | | | |

** dispersed with polyglyceryl-10 Laurate

As can be seen from the above examples, the present invention provides filter systems for microbiome friendly sunscreens having e.g. a SPF of 10 till 50.

### Further Exemplary Formulations

For the formulations 25 to 30, Phase A and part B1 were heated to 80° C. Part B2 was added under stirring into part B1. Phase A was added into Phase B under stirring and homogenized (with an Ultra Turrax type device). Phase C previously prepared was added to the emulsion, which was cooled down under stirring and Phase 0 was added, if present, and the pH was adjusted to >7 with a sodium hydroxide solution.

TABLE 5
Blend formulations 25 to 27.

| | | | 25 | 26 | 27 |
|---|---|---|---|---|---|
| | | | Amount | Amount | Amount |
| | Trade name/tested compound | INCI | [%] | [%] | [%] |
| A | | Glyceryl Stearate SE | 2.00 | 2.00 | 2.00 |
| | Lanette E | Sodium Cetearyl Sulfate | 0.50 | 0.50 | 0.50 |
| | | C12-15 Alkyl benzoate | 10.00 | 10.00 | 10.00 |
| | | Isopropyl palmitate | 10.00 | 10.00 | 10.00 |
| | | C18-38 Alkyl | 1.50 | 1.50 | 1.50 |
| | | Hydroxystearoyl Stearate | | | |
| | Euxyl PE9010 | Phenoxyethanol (and) | 1.00 | 1.00 | 1.00 |
| | | Ethylhexylglycerin | | | |
| | hexyl 2-[4-(diethylamino)-2- | diethylamino hydroxybenzoyl | 2.00 | 2.50 | 3.00 |
| | hydroxybenzoyl]benzoate | hexyl benzoate (DHHB) | | | |
| | 2,4-Bis-{[4-(2-ethyl-hexyloxy)- | bis-ethylhexyloxyphenol | 2.00 | 3.00 | 3.00 |
| | 2-hydroxy]-phenyl}-6-(4- | methoxyphenyl triazine | | | |
| | methoxyphenyl)-1,3,5-triazine | (BEMT) | | | |
| | 4,4′,4″-(1,3,5-triazin-2,4,6- | ethylhexyl triazone (EHT) | 1.00 | 2.00 | 2.50 |
| | triyltriimino)tris-benzoic acid- | | | | |
| | tris(2-ethylhexyl)ester | | | | |
| B1 | | Aqua | Ad | Ad | Ad |
| | | | 100% | 100% | 100% |
| | | Glycerol | 8.00 | 8.00 | 8.00 |
| | | Glucomannan | 0.20 | 0.20 | 0.20 |
| B2 | Rheocare XGN | Xanthan Gum | 0.20 | 0.20 | 0.20 |
| C | Water | Aqua | 5.00 | 5.00 | 8.00 |
| | 2-Phenyl-3H-benzimidazole- | Phenylbenzimidazole | 1.00 | 2.00 | 3.00 |
| | 5-sulfonic acid | Sulfonic Acid (PBSA) | | | |
| | | Sodium hydroxide solution | Qs | Qs | Qs |
| | | | pH >7 | pH >7 | pH >7 |
| D | 2,2′-Methylenebis[6-(2H-1,2,3- | Methylene | — | — | 2.00 |
| | benzotriazol-2-yl)-4-(2,4,4- | Bis-Benzotriazolyl | | | |
| | trimethylpentan-2-yl)phenol] | Tetramethylbutylphenol** | | | |
| | 2,4,6-Tris(biphenyl-4-yl)- | Tris-biphenyl Triazine** | — | 1.00 | 2.00 |
| | 1,3,5-triazin | | | | |

**dispersed with polyglyceryl-10 Laurate

TABLE 6
Blend formulations 28 to 30.

| | | | 28 | 29 | 30 |
|---|---|---|---|---|---|
| | | | Amount | Amount | Amount |
| | Trade name/tested compound | INCI | [%] | [%] | [%] |
| A | | Sucrose Polystearate* | 3.00 | 3.00 | 3.00 |
| | Eumulgin prisma | Disodium Cetearyl | 0.80 | 0.80 | 0.80 |
| | | Sulfosuccinate | | | |
| | Cetiol CC | Dicaprylyl Carbonate | 10.00 | 10.00 | 10.00 |
| | Cetiol Sensoft | Propylheptyl Caprylate | 10.00 | 10.00 | 10.00 |
| | | Oriza Sativa Rice Bran Wax | 2.00 | 2.00 | 2.00 |
| | Euxyl PE9010 | Phenoxyethanol (and) | 1.00 | 1.00 | 1.00 |
| | | Ethylhexylglycerin | | | |
| | hexyl 2-[4-(diethylamino)-2- | diethylamino hydroxybenzoyl | 2.00 | 2.50 | 3.00 |
| | hydroxybenzoyl]benzoate | hexyl benzoate (DHHB) | | | |
| | 2,4-Bis-{[4-(2-ethyl-hexyloxy)- | bis-ethylhexyloxyphenol | 2.00 | 3.00 | 3.00 |
| | 2-hydroxy]-phenyl}-6-(4- | methoxyphenyl triazine | | | |
| | methoxyphenyl)-1,3,5-triazine | (BEMT) | | | |
| | 4,4′,4″-(1,3,5-triazin-2,4,6- | ethylhexyl triazone (EHT) | 1.00 | 1.00 | 1.50 |
| | triyltriimino)tris-benzoic acid- | | | | |
| | tris(2-ethylhexyl)ester | | | | |
| | | Ethylhexyl salicylate | | 2.00 | 3.00 |
| B1 | | Aqua | Ad | Ad | Ad |
| | | | 100% | 100% | 100% |
| | | Glycerol | 7.00 | 7.00 | 7.00 |
| | | microcrystalline cellulose | 0.20 | 0.20 | 0.20 |
| B2 | Rheocare XGN | Xanthan Gum | 0.20 | 0.20 | 0.20 |
| C | Water | Aqua | 5.00 | 5.00 | 8.00 |
| | 2-Phenyl-3H-benzimidazole- | Phenylbenzimidazole | 1.00 | 2.00 | 3.00 |
| | 5-sulfonic acid | Sulfonic Acid (PBSA) | | | |
| | | Sodium hydroxide solution | Qs | Qs | Qs |
| | | | pH >7 | pH >7 | pH >7 |
| D | 2,2′-Methylenebis[6-(2H-1,2,3- | Methylene | — | — | 2.00 |
| | benzotriazol-2-yl)-4-(2,4,4- | Bis-Benzotriazolyl | | | |
| | trimethylpentan-2-yl)phenol] | Tetramethylbutylphenol** | | | |
| | 2,4,6-Tris(biphenyl-4-yl)- | Tris-biphenyl Triazine** | — | 1.00 | 2.00 |
| | 1,3,5-triazin | | | | |

*Emulgade Sucro or Emulgade Sucro Plus
**dispersed with polyglyceryl-10 Laurate

For the formulations 31 to 34, Phase A and part B were heated to 80° C. Phase A was added into Phase B under stirring and homogenized (with an Ultra Turrax type device). Phase C previously prepared was added to the emulsion, which was cooled down under stirring and Phase 0 was added, if present, and the pH was adjusted to >7 with a sodium hydroxide solution.

TABLE 7
Blend formulations 31 to 34.

| | | | 31 | 32 | 33 | 34 |
|---|---|---|---|---|---|---|
| | Trade name/tested | | Amount | Amount | Amount | Amount |
| | compound | INCI | [%] | [%] | [%] | [%] |
| A | | Glyceryl Stearate SE | 2.00 | 2.00 | 2.00 | 2.00 |
| | Eumulgin SG | Sodium Stearoyl | 0.20 | 0.20 | 0.20 | 0.20 |
| | | Glutamate | | | | |
| | Cetiol AB | C12-15 Alkyl benzoate | 10.00 | 10.00 | 10.00 | 10.00 |
| | Cetiol B | Dibutyl Adipate | 10.00 | 10.00 | 10.00 | 10.00 |
| | Lanette 18 | Stearyl Alcohol | 1.50 | 1.50 | 1.50 | 1.50 |
| | | Hydrogenated Castor | 2.00 | 0.50 | 0.50 | 0.50 |
| | | Oil | | | | |
| | Euxyl PE9010 | Phenoxyethanol (and) | 1.00 | 1.00 | 1.00 | 1.00 |
| | | Ethylhexylglycerin | | | | |
| | hexyl 2-[4- | diethylamino | 2.00 | 4.00 | 2.50 | 5.00 |
| | (diethylamino)-2- | hydroxybenzoyl hexyl | | | | |
| | hydroxybenzoyl] | benzoate (DHHB) | | | | |
| | benzoate | | | | | |
| | 2,4-Bis-{[4-(2-ethyl- | bis-ethylhexyloxyphenol | 2.00 | 3.00 | 2.00 | 3.00 |
| | hexyloxy)-2-hydroxy]- | methoxyphenyl triazine | | | | |
| | phenyl}-6-(4- | (BEMT) | | | | |
| | methoxyphenyl)-1,3,5- | | | | | |
| | triazine | | | | | |
| | 4,4′,4″-(1,3,5-triazin- | ethylhexyl triazone | 1.00 | 2.00 | 2.00 | 2.50 |
| | 2,4,6-triyltriimino)tris- | (EHT) | | | | |
| | benzoic acid-tris(2- | | | | | |
| | ethylhexyl)ester | | | | | |
| B | | Aqua | Ad | Ad | Ad | Ad |
| | | | 100% | 100% | 100% | 100% |
| | | Glycerin | 8.00 | 8.00 | 8.00 | 8.00 |
| | | Xanthan Gum | 0.20 | 0.20 | 0.20 | 0.20 |
| C | Water | Aqua | 5.00 | 5.00 | 8.00 | 8.00 |
| | 2-Phenyl-3H- | Phenylbenzimidazole | 1.00 | 2.00 | 2.00 | 3.00 |
| | benzimidazole-5- | Sulfonic Acid (PBSA) | | | | |
| | sulfonic acid | | | | | |
| | | Sodium hydroxide | Qs | Qs | Qs | Qs |
| | | solution | pH >7 | pH >7 | pH >7 | pH >7 |
| D | 2,2′-Methylenebis[6- | Methylene Bis- | — | — | 4.00 | 7.00 |
| | (2H-1,2,3- | Benzotriazolyl | | | | |
| | benzotriazol-2-yl)-4- | Tetramethylbutylphenol* | | | | |
| | (2,4,4-trimethylpentan- | | | | | |
| | 2-yl)phenol] | | | | | |

*dispersed with polyglyceryl-10 Laurate

1.-16. (canceled)

17. A method comprising increasing compatibility of a sunscreen or daily composition with skin microbiome comprising incorporating a sunscreen or daily care composition comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof.

18. The method according to claim 17, wherein the sunscreen or daily care composition comprises hexyl 2-[4-(diethylamino)-2-hydroxybenzoyl]benzoate (INCI diethylamino hydroxybenzoyl hexyl benzoate) and optionally further comprising 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid) Terephthalylidene Dicamphor Sulfonic Acid, and/or drometrizole trisiloxan.

19. The method according to claim 17, wherein the UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), 2-Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS), and mixtures thereof, increases the compatibility of the sunscreen or daily care composition with the population of bacteria selected from the group consisting of Propionibacterium acnes, Propionibacterium granulosum, Propionibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikehum, Corynebacterium tuberculostearicum, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., and mixtures thereof, preferably Staphylococcus epidermidis, on the skin and/or
increases the compatibility of the sunscreen or daily care composition with the population of fungi selected from the group consisting of Malassezia furfur, Malassezia restricta, Malassezia globosa, Candida spp., and mixtures thereof on the skin.

20. The method according to claim 17, wherein the sunscreen or daily care composition further comprises at least one additional UVB filter, preferably selected from the group consisting of 4,4′,4″-(1,3,5-triazin-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoesaure-bis(2-ethylhexyl)ester (INCI diethylhexyl butamido triazone or DBT), dimethicone diethyl benzylmalonate (INCI polysilicone-15), and mixtures thereof, and in particular selected from the group consisting of 4,4′,4″-(1,3,5-triazin-2,4,6-triyltriimino)tris-benzoic acid-tris(2-ethylhexyl)ester (INCI ethylhexyl triazone or EHT), 4,4′-[[6-[[4-[[(1,1-dimethylethyl)amino]carbonyl]phenyl]amino]-1,3,5-triazin-2,4-diyl]diimino]bis-benzoesaure-bis(2-ethylhexyl)ester (INCI diethylhexyl butamido triazone or DBT), and mixtures thereof.

21. The method according to claim 17, wherein the sunscreen or daily care composition further comprises at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine) preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine).

22. The method according to claim 21, wherein the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside) and/or wherein the at least one organic micronized UV filter is dispersed in polyglycerol monoalkyl ester.

23. The method according to claim 17, wherein the sunscreen or daily care composition further comprises at least one additional UVA filter, preferably selected from the group consisting of disodium phenyl dibenzimidazole tetrasulfonate, 1-(4-(1,1-dimethylethyl)phenyl)-3-(4-methoxyphenyl)propane-1,3-dione (INCI Butyl Methoxydibenzoylmethane), and mixtures thereof, in particular 1-(4-(1,1-dimethylethyl)phenyl)-3-(4-methoxyphenyl)propane-1,3-dione (INCI Butyl Methoxydibenzoylmethane).

24. The method according to claim 17, wherein the sunscreen or daily care composition does not contain zinc oxide (ZnO).

25. The method according to claim 17, wherein the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10 wt.-%, preferably from 0.5 to 8 wt.-%, and in particular from 0.5 to 5 wt.-%, based on the total weight of the sunscreen or daily care composition.

26. The method according to claim 17, wherein the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7 wt.-%, preferably from 0.5 to 4 wt. %, based on the total weight of the sunscreen or daily care composition and/or
2-Phenyl-3H-benzimidazole-5-sulfonic acid (INCI Phenylbenzimidazole Sulfonic Acid) in an amount of from 0.1 to 6 wt.-%, preferably from 0.5 to 4 wt.-%, based on the total weight of the sunscreen or daily care composition, and/or
Terephthalylidene Dicamphor Sulfonic Acid (TDSA) in an amount of from 0.1 to 10 wt.-%, preferably from 0.2 to 5 wt.-%, based on the total weight of the sunscreen or daily care composition, and/or
drometrizole trisiloxane (DTS) in an amount of from 0.1 to 10 wt.-%, preferably from 0.2 to 6 wt.-%, based on the total weight of the sunscreen or daily care composition.

27. The method according to claim 17, wherein the sunscreen or daily care composition comprises at least one organic micronized UV filter in an amount of from 0.1 to 10 wt. %, preferably from 0.5 to 5 wt.-%, based on the total weight of the sunscreen or daily care composition.

28. The method according to claim 17, wherein the sunscreen or daily care composition comprises 4,4′,4″-(1,3,5-triazin-2,4,6-triyltriimino)tris-benzoic acid-tris(2-ethylhexyl)ester (INCI ethylhexyl triazone) in an amount of from 0.1 to 5 wt.-%, preferably from 0.5 to 4 wt.-%, based on the total weight of the sunscreen or daily care composition.

29. The method according to claim 17, wherein the sunscreen or daily care composition further comprises a dermatologically acceptable emulsifier, thickener, and/or emollient.

30. The method according to claim 17, wherein the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and/or homosalate.

31. A sunscreen or daily care composition comprising
0.1 to 10 wt.-%, preferably 0.5 to 5 wt.-%, of hexyl 2-[4-(diethylamino)-2-hydroxybenzoyl]benzoate (INCI diethylamino hydroxybenzoyl hexyl benzoate),
0.1 to 8 wt.-%, preferably 0.5 to 4 wt.-%, of 2-Phenyl-3H-benzimidazole-5-sulfonic acid (INCI Phenylbenzimidazole Sulfonic Acid),
0.1 to 5 wt.-%, preferably 0.5 to 3 wt.-%, of 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and
0.1 to 5 wt.-%, preferably 0.1 to 3 wt.-%, of 4,4′,4″-(1,3,5-triazin-2,4,6-triyltriimino)tris-benzoic acid-tris(2-ethylhexyl)ester (INCI ethylhexyl triazone),
0 to 10 wt.-%, preferably 0.1 to 5 wt.-% of 2,2′-methylene bis[6-(2Hbenzotriazol-2-yl)-4-(1,1,3,3-tetramethylbutyl)phenol](INCI Methylene bis-benzotriazolyl tetramethylbutylphenol) and/or 2,4,6-tris(biphenyl-4-yl)-1,3,5-triazine (INCI Tris-Biphenyl triazine),
3 to 15 wt.-%, preferably 5 to 10 wt.-% of glycerol,
each based on the total weight of the sunscreen or daily care composition;
wherein further the sunscreen or daily care composition does not contain a compound selected from the group consisting of ethylhexyl-(2E)-3-(4-methoxyphenyl)acrylate (INCI ethylhexyl methoxy cinnamate), 3-Methylbutyl (2E)-3-(4-methoxyphenyl)prop-2-enoate (INCI Isoamyl p_Methoxycinnamate), ethylhexyl-2-cyano-3,3-diphenyl-acrylate (INCI octocrylene), zinc oxide, and mixtures thereof.

32. Use of a composition (A) comprising a UV filter selected from the group consisting of hexyl 2-[4-(diethylamino)-2-hydroxybenzoyl]benzoate (INCI diethylamino hydroxybenzoyl hexyl benzoate), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), Phenyl-3H-benzimidazole-5-sulfonic acid (INCI Phenylbenzimidazole Sulfonic Acid), Terephthalylidene Dicamphor Sulfonic Acid (TDSA), drometrizole trisiloxane (DTS) and mixtures thereof for producing a sunscreen or daily care composition, which is compatible with the skin microbiome.
